(19) United States
(12) Patent Application Publication
(10) Pub. No.: US 2006/0122120 A1
(43) Pub. Date: Jun. 8, 2006

(54) Method of reducing the effects of Abeta and compositions therefore

(76) Inventors: Jeffrey A. Johnson, Madison, WI (US); Thor D. Stein, Madison, WI (US)

Correspondence Address: QUARLES & BRADY LLP, 411 E. WISCONSIN AVENUE, SUITE 2040, MILWAUKEE, WI, 53202-4497, US

(21) Appl. No.: 11/200,809

(22) Filed: Aug. 10, 2005

Related U.S. Application Data
(60) Provisional application No. 60/600,987, filed on Aug. 11, 2004.
(60) Provisional application No. 60/621,596, filed on Oct. 22, 2004.
(60) Provisional application No. 60/641,683, filed on Jan. 4, 2005.

(51) Int. Cl. A61K 38/10; A61K 38/08; A61K 38/06; C07K 7/08; C07K 7/06

(52) U.S. Cl. 514/14; 514/15; 514/16; 514/17; 514/18; 530/327; 530/328; 530/329; 530/330

(57) ABSTRACT

The invention provides methods and materials related to inhibiting the effects of Aβ such as neuronal cell death and tau phosphorylation. For example, the invention provides polypeptides, compositions containing polypeptides, transgenic animals, and methods for preventing an Aβ effect (e.g., neuronal cell death in a mammal).

### CROSS-REFERENCE TO RELATED APPLICATION

This application claims priority to U.S. provisional applications Ser. No. 60/600,987, filed Aug. 11, 2004, Ser. No. 60/621,596, filed Oct. 22, 2004 and Ser. No. 60/641,683, filed Jan. 4, 2005. All applications are incorporated by reference.

### STATEMENT REGARDING FEDERALLY SPONSORED RESEARCH OR DEVELOPMENT

This invention was made with United States government support awarded by NIH, Grant Nos. ES010042 and ES 008089. The United States government has certain rights in this invention.

### BACKGROUND OF THE INVENTION

In general, the invention relates to methods and materials involved in reducing the effects of Aβ (e.g., neuronal cell death).

Alzheimer's disease (AD) is characterized by β-amyloid (Aβ) accumulation and plaque formation, abnormal phosphorylation and aggregation of the microtubule-associated protein tau, and massive neuronal loss. Mutations identified in hereditary forms of AD as well as abundant animal models and in vitro data strongly implicate Aβ and the polypeptide from which it is derived, the amyloid precursor protein (APP), as the principal factor driving the development of AD (Hardy and Selkoe, Science, 297:353-356 (2002)). Cleavage of APP by β-secretase and γ-secretase produces Aβ. Cleavage of APP by α-secretase occurs within the Aβ sequence and thus precludes Aβ formation while generating an NH₂-terminal, secreted polypeptide termed sAPPα (Esch, et al., Science 248(4959):1122-1124,1990).

Aβ induction of the major AD neuropathologies, including phosphorylation of endogenous tau and in vivo neuronal loss, has yet to be convincingly demonstrated. In fact, though mice overexpressing mutant forms of APP accumulate high levels of Aβ, they do not develop the tau phosphorylation or severe neuronal loss observed in AD (Irizarry et al., J. Neuropathol. Exp. Neurol., 56:965-973 (1997); Irizarry et al., J. Neurosci., 17:7053-7059 (1997)); and Takeuchi et al., Am. J. Pathol., 157:331-339 (2000)).

### BRIEF SUMMARY OF THE INVENTION

In one embodiment, the present invention is an isolated peptide useful for reducing or preventing the effects of Aβ, wherein the peptide comprises the formula A-B—C-D wherein A is selected from the group consisting of amino acid residues D, E, N and Q; wherein B is selected from the group consisting of amino acid residues A, T, S, G and P; wherein C is selected from the group consisting of amino acid residues E, D, N and Q; wherein D is selected from the group consisting of amino acid residues F and Y; wherein the peptide reduces or prevents the effects of Aβ in a mammalian cell and wherein the peptide is between 4-16 residues in length. In a preferred version, the peptide additionally comprises a polypeptide stabilizing unit.

In another embodiment, the peptide additionally comprises amino acid residues at either the carboxy or amino terminal such that the formula of the peptide is X-A-B—C-D-Z wherein X and Z are residues from the sAPPα protein sequence contiguous with DAEF. Preferably, the peptide is selected from the group consisting of DAEF (SEQ ID NO:2), EVKMDAEFR (SEQ ID NO:3), VKMDAEFR (SEQ ID NO:4), EADF (SEQ ID NO:5), SEVKMDAEFR (SEQ ID NO:1), R9DAEF (SEQ ID NO:6), and acDAEF (SEQ ID NO:2).

In another embodiment, the present invention is an isolated peptide useful for reducing and preventing the effects of Aβ, wherein the peptide comprises an isolated 4-16 residue segment of the sAPPα sequence comprising DAEF (residues 597-600).

In another embodiment, the present invention is a method of reducing or preventing the effects of Aβ in a mammalian cell comprising the step of supplying a mammalian cell with an effective amount of a peptide of the present invention such that the effects of Aβ are reduced or prevented.

In another embodiment, the present invention is a method of preventing neuronal cell death comprising the step of supplying a mammalian cell with an effective amount of peptide of the present invention such that neuronal cell death is prevented.

In another embodiment, the present invention is a method of identifying compounds having the ability to reduce the effect of Aβ comprising the steps of contacting brain tissue with a test compound in the presence of an Aβ peptide and measuring the effects of Aβ, preferably additionally comprising the step of comparing the test compound's ability to reduce the effect of Aβ to the ability of a peptide of the present invention to reduce the effect of Aβ.

Unless otherwise defined, all technical and scientific terms used herein have the same meaning as commonly understood by one of ordinary skill in the art to which this invention pertains. Although methods and materials similar or equivalent to those described herein can be used in the practice or testing of the present invention, suitable methods and materials are described below. In case of conflict, the present specification, including definitions, will control. In addition, the materials, methods, and examples are illustrative only and not intended to be limiting.

Other features and advantages of the invention will be apparent from the following detailed description, and from the claims.

### BRIEF DESCRIPTION OF SEVERAL VIEWS OF THE DRAWINGS

The application file contains at least one drawing executed in color. Copies of this patent application publication with color drawing(s) will be provided by the Office upon request and payment of the necessary fee.

FIG. 1 is a bar graph plotting the percent apoptosis for Nissl stained neurons within the neuronal fields of hippocampal slices treated with the indicated polypeptide. Data are presented as mean±SEM of 3 slices per treatment. * indicates a p-value<0.05 compared to 50 μM reverse Aβ treatment; # indicates a p-value<0.05 compared to 25 μM Aβ treatment.

FIG. 2 contains a photograph of an immunoblot of tissue from transgenic and non-transgenic mice using the 6E10 antibody. FIG. 2 also contains a bar graph plotting the relative intensity levels of sAPPα. Values are presented as mean±SEM (n=4). * indicates a p-value<0.05 compared to nontransgenic mice.

FIG. 3 is a self-organized map (SOM). The expression levels of genes and ESTs that were significantly increased (rank ≧9) by 1 nM sAPPα in hippocampal slice cultures were clustered to identify patterns of expression. Expression patterns were examined in vehicle-treated (n=3) and sAPPα-treated (n=3) hippocampal slices, 6-month-old nontransgenic control (n=3) and APP_{Sw} (n=3) mice, and 12-month-old nontransgenic control (n=2) and APP_{Sw} (n=2) mice. Plotted on the ordinate is the average expression level for each cluster of genes (open circles). The other lines (no open circles) indicate the standard deviation for each cluster. Cluster 4 (lower right panel) lists genes upregulated by sAPPα treatment as well as in 6- and 12-month-old APP_{Sw} mice.

FIGS. 4A, B, and C are bar graphs demonstrating that TTR and IGF-2 polypeptides are involved in sAPPα-induced protection against Aβ. The percent death for each treatment was quantified in neuronal fields of live hippocampal slices by counting the number of membrane-permeable, EthD-1 positive cells as well as the number of live cells that stained positively with calcein AM. Data are expressed as mean±SEM (n=3-5 slices per treatment). In FIG. 4A, the live hippocampal slices were treated as indicated. 6E10 is an antibody directed against the COOH-terminal region of sAPPα. The sAPPα fragment is a fragment of the COOH-terminus of sAPPα (SEVKMDAEFR, SEQ ID NO:1) found to mimic the protective effects of sAPPα * indicates a p-value<0.05 compared to 50 μM reverse Aβ; # indicates a p-value<0.01 compared to 50 μM Aβ; ** indicates a p-value<0.05 compared to 1 nM sAPPα+mouse IgG+25 μM Aβ; ## indicates a p-value<0.05 compared to 25 μM Aβ. In FIG. 4B, the live hippocampal slices were treated with vehicle, Aβ, or sAPPα+Aβ together with the indicated antibody (goat IgG, anti-TTR, or anti-IGF-2). * indicates a p-value<0.01 compared to the corresponding vehicle-treated slices; # indicates a p-value<0.01 compared to 1 nM sAPPα+goat IgG+25 μM Aβ. In FIG. 4C, the live hippocampal slices were treated with vehicle, Aβ, or sAPPα+Aβ together with siRNA molecules targeting TTR, IGF-2, or insulin-like growth factor-1 receptor (IGF-1R) sequences. * indicates a p-value<0.05 compared to the corresponding vehicle-treated slices; # indicates a p-value<0.05 compared to 1 nM sAPPα+scrambled GADPH+25 μM Aβ.

FIG. 5A is a bar graph plotting the total number of cells with pyknotic nuclei within the infused CA1 pyramidal neuronal fields of goat IgG (n=4) and anti-TTR (n=4) infused mice. * indicates a p-value<0.01, two-tailed Wilcoxon signed rank test. FIG. 5B is a bar graph plotting the total number of CA1 neurons in goat IgG (n=4) and anti-TTR (n=4) infused mice. * indicates a p-value<0.05, two-tailed Wilcoxon signed rank test.

FIG. 6A is a bar graph plotting the percent death for cortical slices treated as indicated. The percent death for each treatment was quantified in live cortical slices by counting the number of membrane-permeable, EthD-1 positive cells as well as the number of live cells that stained positively with calcein AM. Data are expressed as mean±SEM (n=4 subjects). * indicates a p-value<0.05 compared to 50 μM reverse Aβ; # indicates a p-value<0.05 compared to 50 μM Aβ. FIG. 6B is a bar graph plotting the percent of total cells that were TUNEL positive (% TUNEL) or the percent of NeuN positive cells that were both TUNEL and NeuN positive (% TUNEL/NeuN), for the indicated treatments. Data are from a single subject and are expressed as mean±SEM (n=2-4 slices per treatment). FIG. 6C is a bar graph plotting the percent death for the indicated treatments. The percent death for each treatment was quantified in live cortical slices by counting the number of membrane-permeable, EthD-1 positive cells as well as the number of live cells that stained positively with calcein AM. Data are from three subjects and are expressed as mean±SEM.

FIG. 7 is a bar graph plotting the percent death in hippocampal slice cultures for the indicated treatments. Data are expressed as mean±SEM (n=3-7 slices per treatment). # indicates a p-value<0.01 compared to vehicle; * indicates a p-value<0.01 compared to 25 μM Aβ; unpaired, two-tailed t-test.

FIG. 8 is a bar graph plotting the fold change in relative levels of expression of IGFBP2 in treated and control sides of the hippocampus. The levels were standardized to β-actin levels. The number of samples for the scrambled experiment was three, while the number of samples for the other experiments was four.

FIG. 9 is a bar graph plotting the fold change in relative levels of expression of prolactin receptor in treated and control sides of the hippocampus. The levels were standardized to β-actin levels.

FIG. 10 is a bar graph plotting the fold change in relative levels of expression of IGF2 in treated and control sides of the hippocampus. The levels were standardized to β-actin levels.

FIG. 11 is a diagram of a proposed neuroprotective pathway.

FIGS. 12A and B is a bar graph plotting percent death in hippocampal slice cultures for the indicated treatments.

FIGS. 13A and B is a set of micrographs showing TTR immunostaining following stereotaxic injection of the sAPPα decapeptide into the hippocampus of stereotactic injection of sAPP decamer into hippocampus.

### DETAILED DESCRIPTION OF THE INVENTION

In General

The invention involves methods and materials related to reducing or preventing the effects of Aβ, such as neuronal cell death and tau phosphorylation. For example, the invention provides polypeptides, compositions containing polypeptides, transgenic animals, and methods for preventing an effect of Aβ (e.g., neuronal cell death in a mammal). Such polypeptides and compositions containing polypeptides can be used to provide an amino acid sequence to cells such that an effect of Aβ is reduced or prevented. Reducing or preventing the effects of Aβ can allow scientists to determine the involvement of polypeptides other than Aβ in the development of AD. In addition, reducing or preventing the effects of Aβ can allow clinicians to treat humans suffering from AD or at risk for developing AD. For example, a polypeptide provided herein can be administered to an AD patient such that the neuronal cell death or tau phosphorylation typically observed in untreated AD patients is reduced or prevented in the treated AD patient.

The description of the polypeptides of the present invention below is derived from our discovery of a particular polypeptide fragment of sAPPα that is particularly useful in reducing or preventing effects of Aβ. This polypeptide, SEVKMDAEFR (SEQ ID NO:1), is residues 592-601 of the sAPPα sequence. We have discovered that a peptide of 4 residues within the 10 residue peptide, DAEF, is sufficient in itself to reduce or prevent the effects of Aβ. The residue number system used in this application is based on APP695. See Swiss Prot P05067-4. (See also Kang, et al. Nature 325:733-736, 1987; Lemaire, et al., Nucl. Acids Res. 17:517-522, 1989). This is the predominant isoform in the brain and is therefore the basis of the numbering system we chose.

Polypeptides of the Present Invention

In one embodiment, one aspect of this description features a 4-16 residue polypeptide defined by the following formula:
A-B—C-D (SEQ ID NO:24)

wherein A is selected from the group consisting of amino acid residues D, E and conservative substitutions, such as N and Q;

wherein B is selected from the group consisting of amino acid residues A and conservative substitutions, such as T, S, G, and P;

wherein C is selected from the group consisting of amino acid residues E, D and conservative substitutions, such as N and Q; and

wherein D is selected from the group consisting of amino acid residues F and conservative substitutions, such as Y;

wherein the peptide reduces or prevents the effects of Aβ in a mammalian cell.

By “conservative substitutions” we mean to group amino acids according to the characteristics of the side chains. Predicting a conservative replacement is readily done for the structural regions of large proteins, where there clearly is a good degree of latitude. The best methods derive from studying evolutionary mutations. Two popular methods are PAM250 and Blosum. (See Dayhoff, M. O., et al., Atlas of Protein Sequence and Structure 5(3):345-352, National Biomedical Research Foundation, Washington, 1978; Henikoff, S. and Henikoff, J. G., Proc. Natl. Acad. Sci. USA 89:10915-10919, 1992).

Below is a tabulation of the most conservative substitutions predicted by the PAM250 and Blosum methods for the residues comprising one of the preferred peptides of the present invention.

| | PAM250 | Blosum |
|---|---|---|
| Asp (D) | Glu | Glu/Asn |
| Ala (A) | Thr/Ser/Pro/Gly | Ser |
| Glu (E) | Asp | Asp/Gln |
| Phe (F) | Tyr | Tyr |
| Ser (S) | Ala/Thr/Asn/Pro/Gly | Thr |
| Val (V) | Ile | Ile |
| Lys (K) | Arg | Arg |
| Met (M) | Leu | Ile/Leu |
| Arg (R) | Lys | Lys |

The polypeptide of the present invention is between 4 and 16 amino acid residues in length. Preferably the polypeptide is between 10 and 4 amino acid residues in length. As noted below, one may wish to add additional stabilizing peptides to either end of the molecule. In that embodiment, the total polypeptide may actually be longer than 16 amino acids.

As described below, one may wish to modify the ends of the polypeptide in several ways. These modifications are also within the scope of the polypeptide described above and below. For example, one may wish to provide an acealated end. Additionally, one may wish to modify the polypeptides of the present invention in a manner that does not effect their ability to reduce or prevent the effects of Aβ in a mammalian cell. These trivial modifications are also within the polypeptide of the present invention.

In a preferred form of the present invention, the polypeptide is defined by the formula; A-B—C-D

wherein A is selected from the group consisting of amino acid residues D and E;

wherein B is selected from the group consisting of amino acid residues A;

wherein C is selected from the group consisting of amino acid residues E and D; and

wherein D is selected from the group consisting of amino acid residues F.

In a particularly preferred form of the present invention, the polypeptide consists essentially of A-B—C-D, as defined in either of the embodiments of the invention described above.

In another embodiment of the present invention, the polypeptide is defined by the following formula:
X-A-B—C-D-Z

wherein X and Z are residues from the sAPPα contiguous with DAEF (SEQ ID NO:2). By “contiguous” we mean that the residues naturally abut the naturally occurring DAEF (SEQ ID NO:2) sequence (residues 597-600). For example, the polypeptide EVKM (SEQ ID NO:23)-ABCD would be a peptide of the present invention because the amino acid residues EVKM (residues 593-596) abut the natively occurring DAEF sequence. ABCD-R would be a peptide of the present invention for the same reason.

In preferred embodiments, the polypeptide can comprise or consist of one of the following sAAPα fragments or modified fragments: SEVKMDAEFR (SEQ ID NO:1), SEVKMDAEF (SEQ ID NO:7); EVKMDAEFR (SEQ ID NO:3); VKMDAEFR (SEQ ID NO:4); DAEF (SEQ ID NO:2), acDAEF (SEQ ID NO:2), MEADF (SEQ ID NO:8), EADFR (SEQ ID NO:9), EAEF (SEQ ID NO:10), or EADF (SEQ IDNO:5). The polypeptide can be any 4-16 residue fragment of sAPPα comprising the DAEF (SEQ ID NO:2) segment.

In another embodiment to the present invention, the polypeptide can comprise any one of the previous sequences with conservative amino acid substitutions as described above.

In addition, the polypeptides provided herein can contain a polypeptide stabilizing unit. The term “polypeptide stabilizing unit” as used herein refers to a chemical moiety that increases the stability of a polypeptide within mammalian serum when the chemical moiety is attached covalently or non-covalently to the polypeptide and/or can facilitate the uptake of the polypeptide into a cell.

Numerous proteases have access to molecules within serum and can destroy or metabolize these molecules. A polypeptide stabilizing unit can reduce or prevent the enzymatic degradation of the polypeptides provided herein as well as facilitate their entrance into the brain. A polypeptide stabilizing unit can be a polypeptide that is attached to another polypeptide via a covalent bond, such as an amide bond or peptide bond, or via the interaction between avidin and biotin. The bond may include a bond that is cleaved within the brain, such as —S—S—, an ester, or a stearically hindered ester with a controlled hydrolysis rate.

The stabilizing unit may also be attached via a linker which may a chain of alkyl (methylene), alkoxy (ether), or glycol. In addition, the polypeptide can be attached to the polypeptide stabilizing unit via a polyethylene glycol linker. As above, the linker may include a bond that is cleaved within the brain, such as —S—S—. In these cases, the polypeptide to be stabilized and the polypeptide stabilizing unit can form a larger chimeric polypeptide.

Examples of amino acid sequences that can be used as a polypeptide stabilizing unit include, without limitation, the plasma protein transferrin, fragments of transferrin, an antibody to the transferrin receptor (e.g., OX26 or 8D3), insulin, an antibody to the insulin receptor (e.g., 8314) (Coloma et al., Pharm. Res., 17:266-74 (2000)), IGF-1, IGF-2, and cationised albumin. In some embodiments, a polypeptide stabilizing unit can be a moiety other than a hapten. For example, a polypeptide stabilizing unit can be a moiety other than DNP.

Polypeptide stabilizing units can facilitate uptake of a polypeptide into a cell and/or the brain. Polypeptide stabilizing unit may function to increase the lipophilic-hydrophilic balance and to assist in achieving simple passive diffusion across the lipid membranes of the brain endothelial cells that form the BBB. For example, a long-chain fatty acid, preferably less than 16 carbons, such as stearic acid or a long chain alcohol preferably less than 16 carbons such as steryl alcohol would be preferred. Examples of such polypeptide stabilizing units include, without limitation, a basic domain of the Tat protein of HIV-1 (e.g., Tat₄₉₋₅₇; RKKRRQRRR (SEQ ID NO:11), a 9-mer of L- or D-arginine (R9), or other peptoid analogues such as those containing a six-methylene spacer between the guanidine head group and backbone (Wender et al., PNAS, 97:13003-13008 (2000)). Other amino acid sequences or moieties that can be used as a polypeptide stabilizing unit can be found in PCT/US99/23731; Laras, et al, Org. Biomol. Chem. 3:612-618, 2005; Misra, et al, J. Pharm Pharmaceut. Sci. 6(2):252-273, 2003; Dalpiaz, et al, Science 24:259-269, 2005; Quelever, et al, Org. Biomol. Chem. 3:2450-2457, 2005, and PCT/EP02/02234.

A stabilized unit may exploit receptors (Receptor Mediated Transport—RMT) across the blood brain barrier (Pardridge, NeuroRX2:3-14, 2005). Examples include: transferring or transferin receptor (TfR) antibody, (preferably a fully human MAb); insulin or insulin receptor antibody (preferably a fully human MAb); Type 1 scavenger receptor (SR-VI) (modified LDL); Liposomes (pegylated Immunoliposomes) (Pardridge, Meth. Enzymol. 373:507-528, 2003); nanoparticles (Olivier, NeuroRx 2:108-119, 2005; Olivier, et al., Pharm. Res. 19:1137-1143, 2002); IGF-1; IGF-2; cationized albumin; and Tat protein of HIV.

The polypeptide stabilizing unit might be a substrate for a natural brain transporter to achieve transport-mediated permeation of the peptide across the blood-brain barrier (Tsuji, NeuroRx 2:54-62, 2005), also known as Carrier-Mediated Transport—CMT (Pardridge, supra, 2005), which would exploit the transporters of the Solute Carrier (SLC) gene family (over 100 genes) (Pardridge, supra, 2005). These transporters would include: system L (large, neutral amino acid—F, Y, L) transporter family; hexose (glucose) transporter family; monocarboxylate (short-chain fatty acids) transport family) organic anion transport family; organic cation transport family; ascorbic acid transport family (including certain hexose transporters, Dalpiaz, et al., Eur. J. Pharm. Sci. 24:259-269, 2005; Manfredini, et al., J. Med. Chem. 45:559-562, 2002; Manfredini, et al., Bioorg. Med. Chem. 12:5453-5463, 2004; Quelever, et al., Org. Biomol. Chem. 3:2457-2457, 2005, PCT WO 02/070499); peptide (enkephalin, leptin, ghrelin, thyrotropin-releasing hormone) transport family; and as yet uncharacterized transporters of the SLC gene family.

The polypeptide stabilizing unit might prevent the polypeptide from being pumped out of the brain by a member of the Active Efflux Transport family, including the large family of ATP binding cassette (ABC) proteins, such as the p-glycoprotein (multi-drug resistance; MDR; ABC-B1 gene) pump or other pumps that play a key role in the BBB (Vaalburg, et al., Toxicol. Appl. Pharmacol., 2005; de Boer, et al., Annu. Rev. Pharmacol. Toxicol. 43:629-656, 2003; Fromm, Trends Pharmacol. Sci. 25:423-429, 2004).

Polypeptide stabilizing unit might include a chemical delivery system or redox delivery system, such as dihydronicotinyl moiety (Laras, et al., Org. Biomol. Chem. 3:612-618, 2005) or 1,4-dihydrotrigonellinate moiety (Bodor, Ann. NY Acad. Sci. 507:289-306, 1987; Bodor and Buchwald, Adv. Drug Deliv. Rev. 36:2290-254, 1999; Bodor, et al., Science 257:1698-1700, 1992).

Several studies suggest that proline residues are more resistant to peptidases (Yaron and Naider, Crit. Rev. Biochem. Mol. Biol. 28:31-81, 1993; Vanhoof, et al., Faseb. J. 9:736-744, 1995; Cunningham and O'Connor, Biochim. Biophys. Acta 1343:160-186, 1997). Thus, the polypeptide stabilizing unit can be one or more proline amino acid residues. Such residues can be NH₂-terminal residues, COOH-terminal residues, or both. In fact, one study demonstrated that the addition of the amino acids alanine, proline, proline (APP) or PPA to the NH₂— or COOH-terminal end stabilized a short peptide fragment (Walker, et al., J. Pept. Res. 62:214-226, 2003). The amino acids APP conferred more stability than PP, which conferred more stability than a single P. In addition, protection of the NH₂-terminal end conferred more stability than the COOH-end, consistent with the notion that there are more aminopeptidases than carboxypeptidases. In fact, addition of the amino acids APP conferred more stability than amidation or acetylation and was nearly as effective as cyclization, which completely prevents exopeptidases from accessing a free NH₂-terminus (Walker, supra, 2003). Such stability residues can be N-terminal residues or C-terminal residues. For example, the N-terminus of a polypeptide containing a DAEF sequence can be two proline residues. In some embodiments, a polypeptide can contain both N-terminal and C-terminal proline residues (e.g., PPDAEFPP (SEQ ID NO:12), PDAEFPP (SEQ ID NO:13), PPDAEFP (SEQ ID NO:14), and PDAEFP (SEQ ID NO:15)).

The Swedish mutation in APP (K595 to N and M596 to L) results in increased cleavage by β-secretase at the bond between APP residues 596 and 597. Thus, in those peptides including the β-secretase cleavage site, incorporating this change will simultaneously facilitate production of a smaller neuroprotective peptide containing DAEF (SEQ ID NO:2) and competitively inhibit β-secretase.

A polypeptide stabilizing unit can be a chemical moiety other than an amino acid sequence or an amino acid residue. For example, a polypeptide stabilizing unit can be a poly(ethylene glycol), poly(styrene maleic acid), non-natural amino acids (e.g. arginine analogs and lysine analogs (Kennedy et al., J. Peptide Res., 55:348-358 (2000); Argolyn Bioscience, Inc.), esters (e.g., aromatic benzoyl esters or branched chain tertiary butyl esters), fatty acid or cholesterol ester, an amide group, avidin or streptavidin, or a biotin chemical group that is covalently or non-covalently attached to a polypeptide to be stabilized. In some embodiments, liposomes or immunoliposomes with or without poly(ethylene glycol) inserted into the lipid bilayer (Huwyler et al., PNAS, 93:14164-69 (1996) and Cerletti et al., J. Drug Target, 8:435-46 (2000)) can be used as a polypeptide stabilizing unit and as a method to transport polypeptides into the brain. For example, a liposome containing antibodies to transferrin receptors can be used as a polypeptide stabilizing unit. Modifications such as methylation, including trimethylation of phenylalanine, acetylation, acylation, alkylation, halogenation, and glycosylation can be used to stabilize a polypeptide and increase bioavailability. These and other modifications can function to increase lipid solubility (lipidization) or cationization. Modification of the amino terminus with N-acylation or pyroglutamyl residues can protect the polypeptide from proteolytic cleavage.

The polypeptides of the present invention can be substantially pure. The term “substantially pure” as used herein with reference to a polypeptide means the polypeptide is substantially free of other polypeptides, lipids, carbohydrates, and nucleic acid with which it is naturally associated. For example, a substantially pure polypeptide is any polypeptide that is removed from its natural environment and is at least 60 percent pure. The term “substantially pure” as used herein with reference to a polypeptide also includes chemically synthesized polypeptides and polypeptide compositions. Typically, a substantially pure polypeptide will yield a single major band on a non-reducing polyacrylamide gel.

A vitamin C (ascorbic acid—“AA”) transport systems may be exploited to transport drugs into the brain that would not otherwise cross the BBB (Dalpiaz, et al., Eur. J. Pharm. Sci. 24:259-269, 2005; Manfredini, et al., J. Med. Chem. 45:559-562, 2002; Manfredini, Bioorg. Med. Chem. 12:5453-5463, 2004; Quelever, et al., Org. Biomol. Chem. 3:2450-2457, 2005, PCT WO 02/070499). The advantage of this system is the high concentration of AA-peptide conjugate that may potentially be achieved in the brain. The advantage of the SVCT2 system is the low level of competing natural ascorbic acid ligand in a person on a normal diet and the direct delivery of the conjugate into the DSF bathing the brain. The advantage of the GLUTI system is the very high transport capacity (Tsuji, NeuroRx 2:54-62, 2005), very high surface area of the brain capillaries, relative to the area of the choroids plexus capillaries, providing higher transport capacity and more importantly still, a shorter distance that drugs need to diffuse to gain access to neurons (<50 micrometers) (Pardridge, NeuroRx 2:3-14, 2005), ensuring that all neurons are contacted by the drug. The disadvantage of the GLUT1 system is the relatively high concentration of D-glucose in normoglycemic persons that may compete with the transport of the AA-linker-peptide conjugate (Agus, et al., J. Clin. Invest. 100:2842-2848, 1997). PCT WO 02/070499 describes AA-linker-drug conjugates containing an active substance with a —OH, —SH, or —NH group binding to the carboxyl group of the linker via an ester, thioester or amide bond.

An example of a preferred AA-based polypeptide stabilizing unit comprises ascorbic acid or 6-halo-ascorbic acid or pharmaceutically acceptable derivative and a linker. The linker may contain (i) a moiety with a chemical group to provide a link to the peptide (ii) a chemical group to provide a link to the 5- or 6-OH of AA and (iii) a spacer unit. One or both of the links may be metabolically labile, such as an ester or thioester (so that the neuroprotective peptide may be released in the brain). The linker may be attached to the peptide through the alpha-carboxyl or alpha-amino groups or through any side chain group (for example —OH, —NH2, —SH, —COOH, phenolic —OH). The linker can also be relatively metabolically stable, such as an amide bond. The spacer unit can be a chain of any number of methylenes, but preferably less than 16, optionally containing one or more metabolically-cleavable bonds such as —S—S— such that the neuroprotective peptide attached to part of the linker may be released in the brain. In any situation where part of the linker remains attached to the neuroprotective peptide, this part of the linker must be selected such that the ensemble is biologically active, i.e. neuroprotective, and ideally selected such that the ensemble is more active than the peptide alone.

An example of an AA-based polypeptide stabilizing unit is Ra-AA (attached via an ester bond to the 5- and/or 6-OH of AA) where (1a) R═HO(CH₂)xCO— and a=1 or 2 and x=1 to 16 or (1b) H₂N(CH₂)xCO— and a=1 or 2 and x=1 to 16.

Another example of an AA-based polypeptide stabilizing unit is as above, where (2a) R═HO(CH₂)y-S—S—(CH₂)zCO— and y=1 to 15, z=1−15 and y+z is preferably not greater than 16 or (2b) R═H₂N(CH₂)y-S—S—(CH₂)_{z}CO— and y=1 to 15, z=1−15 and y+z is preferably not greater than 16.

A suitable neuroprotective peptide-polypeptide stabilizing unit would be selected as follows: (i) a range of active neuroprotective peptides or derivatives of the neuroprotective peptide containing any part of the linker that is likely to be metabolically stably attached to the peptide (in the case of the example 1b above it would be —HN(CH₂)xCO₂H or in the case of example 2b, it would be —HN(CH₂)_{y}SH), would be selected by using a suitable assay as described elsewhere in this application (ii) these would be chemically coupled to ascorbic acid using suitable protecting and coupling reagents (iii) the resulting conjugates would be tested in vitro for their ability to be transported across cell membranes by the GLUT1 and SVCT2 transporters (iii) then tested in vivo as described elsewhere in this application and (iv) conjugates with acceptable in vivo potency, activity and duration of action would be selected for additional investigation and further development.

Any method can be used to obtain a substantially pure polypeptide. For example, common polypeptide purification techniques such as affinity chromatography and HPLC as well as polypeptide synthesis techniques can be used. In addition, any material can be used as a source to obtain a substantially pure polypeptide. For example, tissue from wild type or transgenic animals can be used as a source material. In addition, cultured cells engineered to over-express a particular polypeptide of interest can be used to obtain substantially pure polypeptide. A polypeptide can be designed to contain an amino acid sequence that allows the polypeptide to be captured onto an affinity matrix. For example, a tag such as c-myc, hemagglutinin, polyhistidine, or Flag™ tag (Kodak) can be used to aid polypeptide purification. Such tags can be inserted anywhere within the polypeptide including at either the carboxyl or amino termini. Other fusions that could be useful include enzymes that aid in the detection of the polypeptide, such as alkaline phosphatase.

Treatment Methods

In another aspect, the invention features a method for preventing neuronal cell death. The method includes contacting a neuronal cell with a polypeptide, wherein the polypeptide comprises an amino acid sequence of the present invention. The neuronal cell can be a hippocampal cell. As described above, the polypeptide is preferably between 16 and 4 amino acid residues in length, and the polypeptide can contain a polypeptide stabilizing unit.

The polypeptides provided herein prevent neuronal cell death by reducing, inhibiting, or preventing an effect of Aβ. For example, a polypeptide of the present invention can be used to reduce the level of Aβ-induced tau phosphorylation by at least 20 percent. The polypeptides provided herein also can reduce the ability of Aβ to cause an Aβ effect in a mammal. For example, a polypeptide of the present invention can be used to reduce the ability of Aβ to kill cells (e.g., neurons) within a mammal. The reduction in an effect of Aβ or the reduction in the ability of Aβ to cause an Aβ effect can be a complete reduction (e.g., a 100 percent reduction) or an incomplete reduction (e.g., less than 100 percent reduction). For example, in the embodiment the reduction can be a 20 percent or more reduction. In another embodiment, the reduction can be 40 percent.

Aβ effects include, without limitation, tau phosphorylation, neurofibrillary tangle formation, neuronal cell death, neuronal dysfunction, loss of synapses, cellular damage by oxidative stress, and microglia activation. Such effects can be detected or measured using common molecular biology techniques. For example, immunoblotting and immunocytochemistry techniques with anti-phosphorylation antibodies can be used to assess tau phosphorylation (Augustinack et al., Acta Neuropathol., 103:26-35 (2002)). Electron microscopy, thioflavin S histochemistry, and silver staining can be used to detect advanced stages of tau pathology (Greenberg and Davies, PNAS, 87: 5827-31 (1990), Bancher et al., Brain Res., 477: 90-99 (1989), and Uchihara et al., Acta Neuropathologica, 102: 462-6 (2001)). Immunocytochemistry techniques can be used to detect oxidative stress (Abe et al., J. Neurosci. Res., 70:447-50 (2002), and Butterfield and Lauderback, Free Radic. Biol. Med., 32:1050-60 (2002)), microglia activation (Sasaki et al., Acta Neuropathol., 94:316-22 (1997) and Egensperger et al., Brain Pathol., 8: 439-47 (1998)), loss of synapses (Tiraboschi et al., Neurology, 55:1278-83 (2000), Qin et al., Acta Neuropathol., 107:209-15 (2003), and Hatanpaa et al., J. Neuropathol. Exp. Neurol., 58:637-43 (1999)), and neuronal cell death (Su et al., Neuroreport, 5:2529-33 (1994) and Stadelmann et al., Am. J. Pathol., 155:1459-66 (1999)). Measurement of the fluorescence given off by ethidium homodimer when it binds to DNA can be used to detect neuronal cell death within mammals. TUNEL combined with a Nissl stain to look for the morphological features consistent with apoptosis (e.g., chromatin condensation) can detect neurons dying through an apoptotic process (Sheng et al., J. Neuropathol. Exp. Neurol., 57:323-28 (1998) and Pompl et al., Arch Neurol., 60:369-76 (2003)). Electrophysiology can be used to detect deficits in long-term potentiation that can be indicative of neuronal dysfunction (Trinchese et al., Ann. Neurol., 55:801-14 (2004)).

In some embodiments, a polypeptide or composition provided herein can be administered to a mammal (e.g., a mouse, rat, rabbit, monkey, or human) under conditions wherein the degree of an Aβ effect (e.g., amount of neuronal cell death) normally induced by Aβ is reduced. As described herein, the reduction in an effect of Aβ or the reduction in the ability of Aβ to cause an Aβ effect can be a complete reduction (e.g., a 100 percent reduction) or an incomplete reduction (e.g., less than 100 percent reduction. For example, the reduction can be a 20 percent or 40 percent reduction.

The polypeptides or compositions provided herein can be administered by a number of methods depending upon whether local or systemic treatment is desired and upon the area to be treated. For example, a polypeptide or composition provided herein can be administered orally, subcutaneously, intrathecally, intraventricularly, intramuscularly, intraperitoneally, intranasally or intracranially. The administration can be rapid (e.g., by injection) or can occur over a period of time (e.g., by slow infusion or administration of slow release formulations). For treating tissues in the central nervous system, the polypeptides or compositions provided herein can be administered by injection or infusion into the cerebrospinal fluid, preferably with one or more agents capable of promoting penetration across the blood-brain barrier. Examples of such agents include, without limitation, the plasma protein transferrin, an antibody to the transferrin receptor (e.g., OX26), insulin, IGF-1, IGF-2, cationised albumin, a basic domain of the Tat protein of HIV-1 (e.g., Tat₄₉₋₅₇), a 9-mer of L- or D-arginine (R9), or other peptoid analogues such as those containing a six-methylene spacer between the guanidine head group and backbone (Wender et al., PNAS, 97:13003-13008 (2000)). For oral administration, the polypeptides or compositions provided herein can be formulated into powders or granules, suspensions or solutions in water or non-aqueous media, capsules, sachets, or tablets. Such formulations can incorporate thickeners, flavoring agents, diluents, emulsifiers, dispersing aids, or binders.

Referring to intranasal delivery, this is a method of delivering peptides into the bloodstream without the need for injection. With certain drugs it may be sued to bypass the blood-brain barrier and achieve brain penetration via the olfactory and trigeminal nerves. It may use an intranasal liquid formulation as a spray or may be a powder, either may be administered using a mechanical system to give a controlled particle size. Optionally, it can be in a metered dose form, such as a metered aerosol system. (See, Frey, Drug. Deliv. Tech. 2(5):46-49, 2002; DiPietrio and Wolley, Manufact. Chem., pp. 19-22, 2003). The dose may contain one or more excipients such as an aggregation inhibitory agent; a charge-modifying agent; a pH control agent; a degradative enzyme inhibitory agent; a mucolytic or mucus clearing agent; a membrane penetration-enhancing agent, such as a surfactant; a vasodilator agent; a tight junction modulatory agent; a delivery vehicle or carrier.

Implantable drug infusion systems and the ability to place neurosurgical catheters make direct drug delivery to the brain possible. This avoids systemic drug side effects, peripheral drug metabolism and inactivation, serum protein binding, and blood brain barrier penetration (Harbaugh, Psychopharmacol. Bull. 22:106-109, 1986). Such a system may be ideally suited for peptide-based drugs, where systemic stability and BBB penetration are particularly problematic. The cerebrospinal fluid (CSF) is contained in an accessible compartment and baths the entire brain, making it an ideal target for drug delivery. Catheters are routinely placed within the lateral ventricles of patients with hydrocephalus as ventriculoperitoneal shunts that allow CSF to flow from the ventricles into the peritoneum. A ventriculostomy is used to deliver antibiotics such as vancomycin and gentamycin to the CSF of infected patients (Morrison, et al., J. Neurooncol. 11:65-69, 1991). In addition, tPA has been delivered into the lateral ventricles via a ventriculostomy as a treatment for intraventricular hemorrhage (Deutsch et al., Surg. Neurol. 61-460-463, 2004). Intraventricular infusion is also performed with chemotherapeutic agents for the treatment of tumors (Dakhil, et al., Cancer Treat. Rep. 65:401-411, 1981; Fleischhack, et al., Clin. Pharmacokinet. 44:1-31, 2005). Moreover, catheters are placed into the intrathecal space of the lumbar spinal cord and attached to implantable pumps (Medtronic, Minneapolis, Minn.) that deliver baclofen or morphine for the treatment of spasticity and chronic pain.

One preferred method to deliver a polypeptide or any composition herein is to inject or infuse a compound directly into the cerebral ventricles through a ventriculostomy. Dosing can be from 0.01 μg to 1 mg/kg of body weight and can be given once or more daily, weekly, or even less often. Compounds can be delivered into the lateral ventricle by injection or continuously using an implantable pump. The pump can be implanted subcutaneously in the infraclavicular fossa and can deliver a set rate of compound through a catheter to a ventriculostomy reservoir and into a lateral ventricle. The administration of the compound can be isovolumetric, i.e., an amount of CSF equivalent to the volume of compound to be administered is removed before compound injection. Compounds can be dissolved and/or diluted in saline. Alternatively, compounds may be diluted in artificial cerebrospinal fluid (147 mM Na, 2.88 mM K, 127 mM Cl, 1.0 mM phosphate, 1.15 mM Ca, 1.10 mM Mg, 1.10 mM SO₄, 23.19 mM HCO₃, 5,410 mg/L glucose; 300 mOsm/kg).

Any method can be used to formulate and subsequently administer a polypeptide or composition provided herein. Dosing is generally dependent on the severity and responsiveness of the disease state to be treated, with the course of treatment lasting from several days to several months, or until a cure is effected or a diminution of the disease state is achieved. Routine methods can be used to determine optimum dosages, dosing methodologies, and repetition rates. Optimum dosages can vary depending on the relative potency of individual polypeptides, and can generally be estimated based on EC₅₀ values found to be effective in in vitro and/or in vivo animal models. Typically, dosage is from about 0.01 μg to about 100 g per kg of body weight, and can be given once or more daily, weekly, or even less often. Following successful treatment, it may be desirable to have the patient undergo maintenance therapy to prevent the recurrence of the disease state. For example, a preferred oral dose would be 200-4000 mg, preferably divided into four doses per day.

Compositions

The polypeptides provided herein can be formulated into a polypeptide composition that contains additional ingredients. For example, a polypeptide provided herein can be combined with a polypeptide stabilizing molecule. The term “polypeptide stabilizing molecule” as used herein with reference to a polypeptide composition refers to a chemical moiety that increases the stability of the polypeptide within the polypeptide composition when that composition is exposed to mammalian serum. A polypeptide stabilizing molecule can reduce or prevent the enzymatic degradation of the polypeptides provided herein as well as facilitate their entrance into the brain. Examples of polypeptide stabilizing molecules include, without limitation, liposomes, immunoliposomes (e.g., liposomes containing antibodies such as antibodies that bind transferrin), the plasma protein transferrin, fragments of transferrin, an antibody to the transferring receptor (e.g., OX26), insulin, IGF-1, IGF-2, and cationised albumin. Polypeptide stabilizing molecules can facilitate uptake of the polypeptide into a cell and/or the brain. Examples of these include, without limitation, a basic domain of the Tat protein of HIV-1 (e.g., Tat₄₉₋₅₇), a 9-mer of L- or D-arginine (r9), or other peptoid analogues such as those containing a six-methylene spacer between the guanidine head group and backbone (Wender et al., PNAS, 97:13003-13008 (2000)). In some embodiments, a polypeptide stabilizing molecule can be a moiety other than a hapten. For example, a polypeptide stabilizing molecule can be a moiety other than DNP.

A polypeptide stabilizing molecule can be attached (e.g., covalently or non-covalently attached) or unattached to a polypeptide within the polypeptide composition. For example, a composition can contain polypeptide stabilizing molecules that do not attached to the polypeptides within the composition. The composition can contain one or more (e.g., two, three, four, five, six, seven, eight, nine, ten, or more) different polypeptide stabilizing molecules. For example, a composition can contain a DAEF polypeptide and three different polypeptide stabilizing molecules. In some embodiments, the polypeptides of a composition can be identical. In other embodiments, several different polypeptide preparations can be within a composition. For example, a composition can contain two, three, four, five, six, seven, eight, nine, ten, or more polypeptides with each having a different amino acid sequence.

A polypeptide stabilizing molecule can be therapeutically acceptable. The term “therapeutically acceptable” as used herein with respect to a polypeptide stabilizing molecule refers to a polypeptide stabilizing molecule that does not induce significant toxicity to a mammal (e.g., rat, mouse, monkey, or human) when administered to that mammal. Standard testing protocols can be used to determine whether a molecule induces significant toxicity when administered to a mammal.

A composition containing one or more of the polypeptides provided herein can contain one or more pharmaceutically acceptable carriers such as a pharmaceutically acceptable solvent, suspending agent, or any other pharmacologically inert vehicle. Pharmaceutically acceptable carriers can be liquid or solid, and can be selected with the planned manner of administration in mind so as to provide for the desired bulk, consistency, and other pertinent transport and chemical properties. Typical pharmaceutically acceptable carriers include, without limitation, water; saline solution; binding agents (e.g., polyvinylpyrrolidone or hydroxypropyl methylcellulose); fillers (e.g., lactose and other sugars, gelatin, or calcium sulfate); lubricants (e.g., starch, polyethylene glycol, or sodium acetate); disintegrates (e.g., starch or sodium starch glycolate); and wetting agents (e.g., sodium lauryl sulfate).

Pharmaceutical compositions can comprise one or more of the peptides disclosed above. For preparing pharmaceutical compositions from the compounds described by this invention, inert, pharmaceutically acceptable carriers can be either solid or liquid. Solid form preparations include powders, tablets, dispersible granules, capsules, cachets and suppositories. The powders and tablets may be comprised of from about 5 to about 95 percent active compound. Suitable solid carriers are known in the art, e.g. magnesium carbonate, magnesium stearate, talc, sugar or lactose. Tablets, powders, cachets and capsules can be used as solid dosage forms suitable for oral administration. Examples of pharmaceutically acceptable carriers and methods of manufacture for various compositions may be found in A. Gennaro (ed.), Remington's Pharmaceutical Sciences, 18th Edition, (1990), Mack Publishing Co., Easton, Pa.

Liquid form preparations include solutions, suspensions and emulsions. Examples may be water or water-propylene glycol solutions for parenteral injection or addition of sweeteners and opacifiers for oral solutions, suspensions and emulsions. Liquid form preparations may also include solutions for intranasal administration.

Aerosol preparations suitable for inhalation may include solutions and solids in powder form, which may be in combination with a pharmaceutically acceptable carrier, such as an inert compressed gas, e.g. nitrogen. Also included are solid form preparations which are intended to be converted, shortly before use, to liquid form preparations for either oral or parenteral administration. Such liquid forms include solutions, suspensions and emulsions. The compounds of the invention may also be deliverable transdermally. The transdermal compositions can take the form of creams, lotions, aerosols and/or emulsions and can be included in a transdermal patch of the matrix or reservoir type as are conventional in the art for this purpose. The compounds of the invention may also be deliverable subcutaneously.

Preferably, the pharmaceutical preparation is in a unit dosage form. In such form, the preparation is subdivided into suitably sized unit doses containing appropriate quantities of the active compound, e.g., an effective amount to achieve the desired purpose. The quantity of active compound in a unit dose of preparation may be varied or adjusted from about 0.01 mg to about 1000 mg, preferably from about 0.01 mg to about 750 mg, more preferably from about 0.01 mg to about 500 mg, and most preferably from about 0.01 mg to about 250 mg, according to the particular application. The actual dosage employed may be varied depending upon the requirements of the patient and the severity of the condition being treated. Determination of the proper dosage regimen for a particular situation is within the skill of the art. For convenience, the total daily dosage may be divided and administered in portions during the day as required. The amount and frequency of administration of the compounds of the invention and/or the pharmaceutically acceptable salts thereof will be regulated according to the judgment of the attending clinician considering such factors as age, condition and size of the patient as well as severity of the symptoms being treated. A typical recommended daily dosage regimen for oral administration can range from about 0.04 mg/day to about 4000 mg/day, in one to four divided doses.

Identifying Compounds

The invention provides methods and materials for identifying compounds having the ability to reduce an effect of Aβ such as those effects described herein. For example, the methods and materials provided herein can be used to identify a compound capable of reducing the level of neuronal cell death induced by Aβ. In one embodiment, cells (e.g., brain tissue or brain slices) can be contacted with a test compound in the presence of an Aβ peptide, and the level of an effect of Aβ can be measured. This measured level can be compared to the level measured using control cells contacted with the Aβ peptide in the absence of the test compound. If less of an Aβ effect (e.g., neuronal cell death) is observed with the cells contacted with the test compound and Aβ peptide as compared to that observed with the control cells, then the test compound can have the ability to reduce an effect of Aβ.

A positive control for such a reduction can be cells contacted with a DAEF-containing polypeptide (e.g., SEVKMDAEFR (SEQ ID NO: 1), SEVKMDAEF (SEQ ID NO:7), EVKMDAEFR (SEQ ID NO:3), VKMDAEFR (SEQ ID NO:4), DAEF (SEQ ID NO:2) or any of the polypeptides of the present invention) and an Aβ peptide. Other controls can include untreated cells and/or cells treated with β-amyloid₁₋₄₂ or reverse Aβ (Aβ₄₂₋₁). In some embodiments, test compounds are evaluated in brain slice culture for their abilities to induce effects comparable to those of sAPPα, SEVKMDAEFR, or DAEF.

Any test compound can be used. For example, a test compound can be a polypeptide, lipid, ester, triglyceride, steroid, fatty acid, or small molecule. In some embodiments, the test compound can be a molecule designed to mimic the structure and/or function of a polypeptide such as a DAEF-containing polypeptide (e.g., SEVKMDAEFR (SEQ ID NO:1), SEVKMDAEF (SEQ ID NO:7), EVKMDAEFR (SEQ ID NO:3), VKMDAEFR (SEQ ID NO:4), or DAEF (SEQ ID NO:2)). For example, the test compound can be a peptidomimetic of a DAEF-containing polypeptide. Such a peptidomimetic can contain amide bond isoteres and/or other peptide backbone modifications. For example, heteroatoms can be used to interfere with proteolytic degradation. In some cased, a peptidomimetic can have secondary structural characteristics such as a helices and/or β sheets. See, e.g., Gellman, S H, Acc. Chem. Res., 31:173-180 (1998). Any method can be used to design a candidate peptidomimetic compound including those reviewed elsewhere. (Fisher, P M, Curr. Protein Pept. Sci., 4 (5):339-56 (2003) and Patch and Barron, Curr. Opinion Chem. Biol., 6:872-877 (2002)). In some embodiments, combinatorial libraries of numerous peptidomimetic compounds can be generated. In these cases, high throughput assays based on, for example, a calorimetric or fluorescent readout can be used to identify compounds having the ability to reduce an effect of Aβ. In other cases, a library of test compounds can be provided in the form of an array and subjected to high throughput screening to identify compounds. See, e.g., Goodman et al., Biopolymers, 60:229-245 (2001) and al-Obeidi et al., Mol. Biotechnol., 9:205-223 (1998).

Test compounds that can reduce or prevent an effect of Aβ or can reduce the ability of Aβ to cause an Aβ effect in a mammal can be obtained using common molecular biology techniques. For example, the techniques provided herein for detecting or measuring an Aβ effect can be used to identify polypeptides having the ability to reduce an Aβ effect. In one embodiment, cells (e.g., cultured neurons) are pre-treated with a test polypeptide followed by treatment with Aβ. The treated cells are then assessed for cell death. If the cells pre-treated with the test polypeptide exhibit less cell death than control cells not pre-treated with the test polypeptide, then the test polypeptide can be a polypeptide that reduces the ability of Aβ to induce an Aβ effect.

Each amino acid residue of a test polypeptide provided herein can be one of the twenty conventional amino acid residues. In some embodiments, the polypeptides can contain one or more modified amino acid residues or any other chemical structure that can be incorporated into a polypeptide including, without limitation, ornithine, citrulline, ε-aminohexanoic acid, hydroxylated amino acids (e.g., 3-hydroxyproline, 4-hydroxyproline, (5R)-5-hydroxy-L-lysine, allo-hydroxylysine, or 5-hydroxy-L-norvaline), glycosylated amino acids (e.g., amino acids containing D-glucose, D-galactose, D-mannose, D-glucosamine, D-galactosamine, or combinations thereof), 2-aminoadipic acid, 3-aminoadipic acid, β-alanine or β-aminopropionic acid, 2-aminobutyric acid, 4-aminobutyric acid, 6-aminocaproic acid, 2-aminoheptanoic acid, 2-aminoisobutyric acid, 3-aminoisobutyric acid, 2-aminopimelic acid, 2,4-diaminobutyric acid, desmosine, 2,2-diaminopimelic acid, 2,3-diaminopropionic acid, N-ethylglycine, N-ethylasparagine, isodesmosin, allo-isoleucine, N-methylglycine, N-methylisoleucine, 6-N-methyllysine, N-methylvaline, norvaline, norleucine, ornithine modifications of arginine, citrulline modifications of arginine, β-D-galactopyranosyl-5-hydroxy-L-lysine with single or multiple deoxygenations, and 2-O-α-D-glucopyranosly-β-D-galactopyranosyl-5-hydroxy-L-lysine with single or multiple deoxygenations. In addition, one or more hydroxyl groups of an amino acid residue or chemical structure can be replaced with fluorine. For example, the hydroxy group of 3-hydroxyproline can be replaced with fluorine to create 3-fluoroproline, or the hydroxy group of 4-hydroxyproline can be replaced with fluorine to create 4-fluoroproline. Further, amino acid residues or chemical structures can have C— or S— or O-glycosidic linkages. A single polypeptide can contain any combination of such amino acid residues and chemical structures. For example, a single polypeptide can contain twelve conventional amino acid residues, eight hydroxylated amino acids, two glycosylated amino acids, and one ornithine in any order.

Typically, a test polypeptide provided herein contains amino acid residues connected by amide bonds (—CONH—). In some embodiments, a polypeptide can contain amino acid residues or chemical structures connected by other bond including, without limitation, modified amide bonds such as those modified by N-methylation (—CONMe-), N-alkylation (—CONR—), or reduction (—CH₂NH—) as well as isosteres bonds such as methylene ether bonds (—CH₂O—), methylene thioether bonds (—CH₂S—), vinyl group bonds (—CH═CH—), ethylene group bonds (—CH₂CH₂—), ketomethylene group bonds (—COCH₂—), thioamide bonds (—CSNH—), and sulfone bonds (—CH₂SO—). A single polypeptide can contain a sequence of amino acid residues or chemical structures connected by any combination of bonds. For example, a single polypeptide can contain a sequence of amino acid residues connected exclusively by amide bonds or by a combination of amide bonds, methylene ether bonds, and sulfone bonds.

The test polypeptides provided herein can be linear polypeptides such that they have free N— and C-termini. The polypeptides can be engineered to contain disulfide bonds or can be designed to be cyclized as described elsewhere (Egleton et al., Peptides 18:1431-39 (1997) and Iwai and Pluckthun, FEBS Lett., 459(2):166-72 (1999)). For example, a polypeptide containing a DAEF sequence can contain two or more (e.g., 2, 3, 4, 5, 6, 7, 8, 9, 10, or more) cysteine residues such that one or more disulfide bonds are formed within the polypeptide. Polypeptides containing a disulfide bond can be more stable to degradation than similar polypeptides lacking a disulfide bond. Two or more polypeptides can be linked by a hydrazide bridge. A hydrazide bridge can protect against degradation by carboxy peptidase activity. Polypeptides can contain a disulfide bridge (e.g., via two D-penicillamine residues) such that the polypeptide is cyclized. Cyclized polypeptides can be more stable to degradation than similar polypeptides that are not cyclized.

The test polypeptides provided herein can contain additional amino acid sequences including those commonly used as tags (e.g., poly-histidine tags, myc tags, GFP tags, and GST tags). For example, a 50 amino acid fragment of an sAPPα polypeptide containing a DAEF sequence can contain the amino acid sequence of a fluorescent polypeptide (e.g., GFP).

Any type of cell can be used. For example, in one preferred embodiment neuronal cell cultures or brain tissue samples (e.g., brain slices) can be used. In addition, the methods can be performed in vivo or in vitro. For example, neuronal cells within a mouse can be contacted with a test compound by injecting the test compound into the mouse's brain.

The Aβ peptide can be any type of Aβ peptide such a human β-amyloid₁₋₄₂. In addition, any method can be used to contact cells with an Aβ peptide. For example, Aβ peptide can be administered to cells in culture. In some embodiments, cells can be contacted with an Aβ peptide that is expressed by cells containing nucleic acid encoding the Aβ peptide. For example, neuronal cells can be designed to contain nucleic acid encoding an APP sequence known to release Aβ peptide. Any method can be used to assess a test compound's ability to reduce an effect of Aβ. For example, staining techniques such as those described herein can be used to assess cell death.

The methods and materials provided herein also can be used to identify a compound capable of increasing the expression of a neuroprotective polypeptide such as TTR and IGF-2. For example, cells (e.g., brain tissue or brain slices) can be contacted with a test compound, and the expression level of nucleic acid encoding the neuroprotective polypeptide can be determined by measuring the polypeptide levels and/or mRNA levels. Any method can be used to measure polypeptide levels including, without limitation, ELISA techniques, antibody staining techniques, and biological activity assays. In addition, any method can be used to measure mRNA levels including, without limitation, RT-PCR techniques and expression array techniques.

The level of polypeptide or mRNA expression can compared to the polypeptide or mRNA expression level determined for control cells not contacted with the test compound. If less polypeptide or mRNA expression is observed with the cells contacted with the test compound as compared to that observed with control brain tissue, then the test compound can have the ability to reduce an effect of Aβ. A positive control for such a reduction can be cells contacted with a DAEF-containing polypeptide (e.g., SEVKMDAEFR (SEQ ID NO:1), SEVKMDAEF (SEQ ID NO:7), EVKMDAEFR (SEQ ID NO:3), VKMDAEFR (SEQ ID NO:4), or DAEF (SEQ ID NO:2)). Other controls can include untreated cells (e.g., untreated brain tissue).

The effectiveness of a test compound to increase the expression level of nucleic acid encoding a neuroprotective polypeptide can be determined by comparing the expression level observed with cells contacted with the test compound with the expression level observed with cells contacted with a DAEF-containing polypeptide. An equivalent or greater level of expression observed with cells contacted with the test compound as compared to that observed with cells contacted with the DAEF-containing polypeptide can indicate that the test compound is a potent activator of neuroprotective polypeptide expression.

The methods and material provided herein can be used to identify a compound capable of increasing or decreasing expression of a polypeptide that is capable of having its expression altered by cells in response to the presence of sAPPα. For example, cells (e.g., neuronal cells) can be contacted with a test compound and assessed to determine whether or not altered (e.g., increased or decreased) expression of a polypeptide that is capable of having its expression altered by cells in response to the presence of sAPPα is exhibited as compared to the expression of that polypeptide by control cells not contacted with the test compound. Examples of such polypeptides include, without limitation, TTR polypeptides and IGF-2 polypeptides. An increase or decrease in expression of a polypeptide can be determined by measuring polypeptide levels and/or mRNA levels.

### EXAMPLES

### Example 1

### Methods and Materials

Animals

Tg2576 mice were created as described previously Hsiao et al., Science, 274:99-102 (1996)). Briefly, they contain the human amyloid precursor protein 695 with the double mutation K670N and M671L (Swedish mutation) and driven by the prion protein promoter. In this study, transgenic and nontransgenic control mice were generated from C57B6/SJL N2 generation Tg2576 mice backcrossed to C57B6/SJL breeders. Mice were sacrificed at 12 and 18 months of age.

Hippocampal Organotypic Cultures

Pups from C57B6/SJL mice were anaesthetized and decapitated at postnatal day 15. The brain was removed and placed in ice-cold dissecting medium (50% Minimum Essential Medium (Invitrogen, Carlsbad, Calif.), 50% Hank's Balanced Salt Solution (Invitrogen), 25 mM HEPES, and 36 mM glucose). Both hippocampi were dissected under a dissecting microscope and cut at 400 μm on a Mcllwain tissue chopper. Slices were separated and placed on filter inserts with a 30 mm diameter and a 0.4 μM filter pore size (Millipore, Billerica, Mass.) held in 6-well culture plates. For the first 3 days, slices were kept in Neurobasal media with B-27 supplement, 1 mM glutamine, and 1% penicillin/streptomycin (Invitrogen). Subsequently, the media was changed every 3 days using media without antibiotics. Organotypic cultures were maintained in a humidified incubator at 37° C. in 5% O₂ and 5% CO₂ for 14 to 21 days.

Cell death and viability was determined on live slices using the fluorescent probes from Molecular Probes' Live/Dead Kit. After a 40 minute incubation in calcein AM (1:400) and ethidium homodimer (1:1000) in phosphate buffered saline, slices were visualized at 200× magnification with an inverted Nikon Diaphot 200 microscope using the Bio-Rad MRC-1024 Laser Scanning Confocal system (Hercules, Calif.). Random images of the neuronal fields were captured at the emission spectrum of each probe, and Scion Image software (Frederick, Md.) was used to quantify the number of live and dead cells. At least three areas including a neuronal field were imaged and used to determine the percent death for each slice. The percent death was calculated as the number of EthD-1 positive cells divided by the total number of EthD-1 and calcein AM positive cells.

Detection of Cell Death by TUNEL and Nissl Staining

Hippocampal slice cryosections from 3 animals per treatment were stained with terminal deoxynucleotidyl transferase-mediated, dUTP nick end labeling (TUNEL). Cells with DNA fragmentation were determined by the terminal deoxynucleotidyl transferase incorporation of fluorescein isothiocyanate (FITC)-12-dUTP into DNA (In Situ Cell Death Detection kit, Roche Biochem, Indianapolis, Ind.). Other sections were stained with cresyl violet acetate, and the neurons with healthy nuclei as well as the neurons with condensed and clumped chromatin were counted within the neuronal fields.

Immunohistochemistry

Mice were killed with CO₂ and immediately perfused through the heart with phosphate buffered saline (PBS). The right hemispheres were fixed in 4% paraformaldehyde (PFA) overnight, sunk in 30% sucrose, and frozen in OCT embedding medium. Hippocampal slices were fixed in 4% PFA for 20 minutes, incubated in 30% sucrose overnight, and frozen in OCT embedding medium. Frozen sections with a width of 10 μm were taken through the hippocampus. Post-mortem human tissue was formalin fixed, cut at 10 μm, and boiled for 10 minutes in a 10 mM Tris buffer, pH 1 for antigen retrieval. IGF-2 and TTR were detected with a 1:200 dilution of the polyclonal antibody against IGF-2 (F-20) or TTR (C-20; Santa Cruz Biotechnology, Santa Cruz, Calif.). Phospho-BAD (Ser112; Cell Signaling, Beverly, Mass.), and BAD (Stressgen, Victoria, British Columbia) were detected with a 1:250 dilution of the respective polyclonal antibody. Phosphorylated tau was detected with the monoclonal AT8 antibody (1:200; Research Diagnostics, Flanders, N.J.) and anti-phospho-tau(Thr231) (1:500; Calbiochem, San Diego, Calif.). As a control pre-immune rabbit or mouse IgG (Vector Laboratories, Burlingame, Calif.) or goat IgG (Santa Cruz) was used in place of the primary antibody. A biotinylated anti-goat IgG, anti-rabbit IgG, or anti-mouse IgG was used as the secondary antibody. Finally, the Vectastain Elite ABC kit (Vector) and tyramide conjugated to either Alexa Fluor 488 or 568 (Molecular Probes, Eugene, Oreg.) were used to visualize the antibody staining. Anti-NeuN (1:250; Chemicon International, Temecula, Calif.) and 4G8 (1:250; Signet, Dedham, Mass.) together with an anti-mouse IgG secondary antibody conjugated to Alexa Fluor 488 were used to detect NeuN and Aβ. The DNA binding dyes ToPro3 (Molecular Probes) or Hoechst 33258 (Sigma, St. Louis, Mo.) were used to visualize nuclei. Sections were imaged using either the Bio-Rad Laser Scanning Confocal system or epifluorescence (Zeiss, Thornwood, N.Y.). Representative figures were obtained from 3 APP_{Sw} mice and 3 non-transgenic controls, 3 animals per treatment for hippocampal slices, or from 4 goat IgG and 4 anti-TTR antibody infused mice.

Electron Microscopy

Following treatment with 50 μM Aβ or reverse Aβ for 24 hours, hippocampal slice cultures were fixed with a mixture of 2% PFA/2.5% glutaraldehyde in 0.1 M Sorensons phosphate buffer (pH 7.4) for 1 hour and then post fixed for 1 hour in 2% osmium tetroxide in the same buffer. The slices were then dehydrated in an ethanol series and embedded in Epon epoxy resin. Ultrathin sections were cut on a Reichert-Jung microtome, placed on copper grids, and stained with routine concentrations of uranyl acetate and lead citrate. Micrographs were taken with a Philips CM120 electron microscope.

Treatments

β-amyloid₁₋₄₂ and reverse Aβ (Aβ₄₂₋₁) were obtained from BACHEM (Torrance, Calif.). These polypeptides were dissolved in 0.1% NH₃OH at 222 μM. For treatments, Aβ was aggregated at 50 μM in Neurobasal media with B-27 supplement and 1 mM glutamine at 37° C. for 24 hours. Then, the slice culture media was removed, and the slices were treated with 1 mL of 25 μM or 50 μM Aβ, reverse Aβ, or vehicle for 24 hours. Human TTR (Calbiochem) or IGF-2 (Peprotech, Rocky Hill, N.J.) was added to slices together with the Aβ treatment at final concentrations of 3 μM (TTR) or 500 nM (IGF-2). sAPPα was prepared as described previously (Mattson et al., Neuron, 10:243-254 (1993)).

Unless otherwise stated, all sAPPα treatments were performed at 1 nM 48 hours prior to the addition of Aβ. Antibodies against TTR (C-20) or IGF-2 (F-20; 4 μg/mL; Santa Cruz) or sAPPα (6E10; 10 μg/mL; Chemicon) were added to slices together with the sAPPα treatment. Goat IgG (4 μg/mL; Santa Cruz) or mouse IgG₁ (10 μg/mL; Chemicon) was added as a control. The 10 amino acid fragment of the carboxyl-terminal region of sAPPα (592-601 of APP695; SEVKMDAEFR; SEQ ID NO:1; Sigma) was added according to the same protocol for sAPPα.

SiRNA's were created based on the mouse mRNA sequences using the Ambion Silencer siRNA Construction Kit (Austin, Tex.). Several siRNA's were created by in vitro transcription for each silenced gene. Transfection was performed for 4 hours at 37° C. prior to vehicle or sAPPα treatment using 25 nM siRNA combined with the polyamines in the Ambion siPORT Amine Transfection Agent. Transfections were performed in Neurobasal media with 1 mM glutamine (Invitrogen). The successful mRNA target sequences are as follows, TTR: 5′-AATCCAAATGTCCTCTGATGG-3′ (SEQ ID NO:16) and 5′-AACTGGACACCAAATCGTACT-3′ (SEQ ID NO:17); IGF-2: 5′-AA-GGGGATAGAGATGTGAGAG-3′ (SEQ ID NO: 18) and 5′-AAATTATGTGGT-AATTCTGCA-3′ (SEQ ID NO:19); IGF-1R: 5′-AACGACTATCAGCAGCTGAAG-3′ (SEQ ID NO:20) and 5′-AACAGCTGGAACATGGTGGAT-3′ (SEQ ID NO:21).

Microarray Analysis

Total RNA was extracted from hippocampal slices of male nontransgenic mice treated with either vehicle or 1 nM sAPPα for 24 hours. Total RNA was isolated with Trizol (Invitrogen, Carlsbad, Calif.) and used to synthesize cRNA as described previously (Stein and Johnson, J. Neurosci., 22:7380-7388 (2002)).

Fifteen μg of fragmented cRNA was hybridized for 16 hours at 45° C. to a MG-U74Av2 array (Affymetrix, Santa Clara, Calif.). Affymetrix Microarray Suite 5.0 was used to scan and analyze the relative abundance of each gene from the intensity signal value. Significantly changed genes were determined using the Wilcoxon signed rank test for each comparison. Probe sets with p-values<0.01 were called Increased/Decreased; probe sets with p-values in the range 0.01<p-value<0.05 were called Marginally Increased/Decreased; and the remaining probe sets were called No Change. An additional level of ranking was used to incorporate multiple comparisons such that No Change=0, Marginal Increase/Decrease=1/−1, and Increase/Decrease=2/−2 (Li and Johnson, Physiol. Genomics, 9:137-144 (2002)). The final rank equaled the sum of the ranks from the 9 comparisons, and the value varied from −18 to 18 for the 3×3 comparison in hippocampal slices. The cutoff values for the final determination of increased or decreased gene expression were set as rank ≧9 and FC≧1.2 for increased genes and rank ≦−9, and FC≦−1.2 for decreased genes. Intensity values for gene expression were normalized to mean=0 and variance=1 and clustered using the self-organized map (SOM) algorithm from the Affymetrix Data Mining Tool.

Infusions

Eighteen-month-old APP_{Sw} mice were deeply anaesthetized with an isoflurane gas anesthesia system and placed in a stereotaxic apparatus (Stoelting, Wood Dale, Ill.). An incision was made to expose the cranium, and a Dremel drill was used to drill through the skull. Cannulas from the Alzet Brain Infusion Kit (Alzet, Cupertino, Calif.) were stereotaxically implanted within the right hippocampi of APP_{Sw} mice (coordinates relative to Bregma: anteroposterior=−2.7 mm, mediolateral=−3.0 mm, and dorsoventral=−3.0 mm) and cemented in place with cyanoacrylate. Osmotic pumps (Alzet) containing 200 μL of 100 μg/mL goat IgG or anti-TTR antibody (C-20, Santa Cruz) were inserted subcutaneously in the mid-scapular region (flow rate, 0.5 μL/hour). Treatments were diluted in artificial CSF containing 150 mM NaCl, 1.8 mM CaCl₂, 1.2 mM MgSO₄, 2.0 mM K₂HPO₄, and 10.0 mM glucose, pH 7.4. The scalps were sutured, and mice were returned to their home cages. After 2 weeks, the goat IgG and anti-TTR infused mice were sacrificed and immediately perfused through the heart with ice-cold PBS followed by 4% PFA.

Stereology

Four goat IgG and 4 anti-TTR antibody infused mice were sectioned at a width of 50 μm through the entire hippocampus. Every sixth section was stained with cresyl violet, and neurons with healthy and intact nuclei were counted within the CA1 pyramidal neuronal field using the optical fractionator technique (West and Gundersen, 1990). Cells within the CA1 pyramidal neuronal field that possessed condensed chromatin were counted separately. Counts were made at 1000× magnification on a Zeiss Axioplan 2 microscope (Gottingen, Germany). Each optical dissector consisted of a 30×30 μm counting frame with extended exclusion lines, a height of 19 μm, and top and bottom guard zones of 3 μm. Total neuron and pyknotic cell counts were obtained from a systematic random sampling of the entire CA1 using the Microbrightfield Stereo Investigator software (Colchester, Vt.).

Statistical Analysis

All the experimental data shown were repeated at least three times. Results are expressed as mean±SEM. Unless otherwise stated, statistical significance was determined using a two-tailed, unpaired student's t test, and a p-value<0.05 was considered significant.

### Example 2

### Aβ Induces Neuronal Death in Organotypic Hippocampal Cultures

Organotypic hippocampal cultures maintained the architecture of the hippocampus and contained synaptic connections with mature synaptic properties, including long-term potentiation as described elsewhere (Bahr, J. Neurosci. Res., 42:294-305 (1995) and Muller et al., Brain Res. Dev. Brain Res., 71:93-100 (1993)). Compared to dissociated cultures, which are typically from embryonic brain, slices represent a more relevant model of the intact adult brain. Therefore, this model was used to study Aβ-induced neuronal toxicity and protection by sAPPα. After 2 weeks in culture, the cornu ammonis (CA) neuronal fields were preserved and stained positively for the postmitotic neuronal marker NeuN.

Ethidium homodimer (EthD-1) is a membrane impermeable DNA-binding dye that is excluded from live cells with an intact plasma membrane. Calcein AM is a cell-permeant dye that fluoresces in live cells with a functional intracellular esterase. When live slices treated with reverse Aβ were incubated with EthD-1 and calcein AM, many neurons in the hippocampal neuronal fields stained positively for calcein AM while only a few stained positively for EthD-1. Treatment with either 25 μM or 50 μM Aβ, however, resulted in a dramatic increase in the number of neurons that lost membrane integrity (EthD-1 positive).

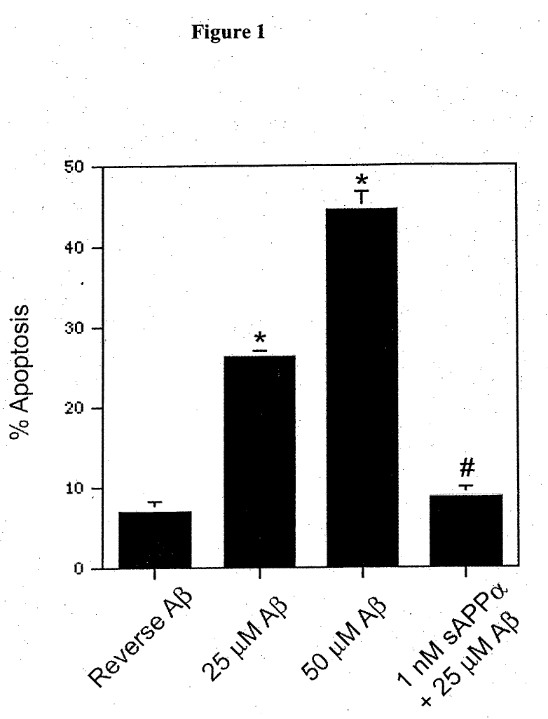

Consistent with neuronal death, treatment with either 25 μM or 50 μM Aβ, but not reverse Aβ, resulted in DNA strand breaks as indicated by TUNEL. The transmission image from the laser scanning confocal system demonstrated that the TUNEL positive cells were located within the neuronal fields. Finally, the percent of apoptosis was measured within the CA neuronal fields of Nissl-stained slices (FIG. 1). In healthy neurons, chromatin was dispersed and did not stain intensely with cresyl violet. However, intense staining was observed in dying cells that have undergone chromatin condensation, a key feature of apoptosis. Treatment with 25 μM or 50 μM Aβ resulted in a significant increase in the percent of neurons with chromatin condensation. Pretreatment with 1 nM sAPPα for 48 hours prevented the Aβ-induced TUNEL staining and nuclear pyknosis (FIG. 1).

### Example 3

### Aβ Induces Tau Phosphorylation

The monoclonal antibody AT8 recognizes phosphorylated tau that is sometimes aggregated as paired helical filaments (PHFs). The epitope includes phosphorylated serine at amino acid 202. Many neurons within AD patients contain PHFs recognized by the AT8 antibody. Vehicle and reverse Aβ-treated hippocampal slices exhibited AT8 staining of some cells around the hilus and within the stratum radiatum. Some of these cells also stained positively when mouse IgG was used as the primary antibody, suggesting this staining may be due the presence of a non-specific antigen or the expression of a mouse IgG-like protein. However, no staining within the hippocampal neuronal fields occurred with a control mouse IgG. Staining with the AT8 antibody resulted in few positively stained neurons in the CA or dentate gyrus hippocampal neuronal fields of vehicle or reverse Aβ-treated slices. In contrast, when treated with 25 μM Aβ or 50 μM Aβ, many neurons within the hippocampal neuronal fields were AT8 positive. These neurons also stained positively for EthD-1, indicating Aβ-induced cellular damage. AT8 staining revealed a neuronal cell body and dendritic redistribution of tau resembling the tau pathology that occurs in AD patients. Further, many AT8 positive neurons demonstrated beaded processes consistent with neuronal degeneration. Pretreatment with sAPPα prevented the Aβ-induced AT8 staining within the hippocampal neuronal fields.

No vehicle or reverse Aβ-treated slices contained neurons positive for tau phosphorylated at threonine 231. In slices treated with 25 μM Aβ or 50 μM Aβ, however, several neurons stained positively for phospho-tau(Thr-231) in their cell bodies and processes. These neurons also possessed a diffuse NeuN staining and pyknotic nuclei, indicative of neuronal degeneration. Pretreatment with 1 nM sAPPα prevented the Aβ-induced accumulation of phospho-tau(Thr-231). An immunoblot for phosph-tau(Thr-231) was performed on slices treated with 50 μM reverse Aβ, 50 μM Aβ, and 1 nM sAPPα+50 μM Aβ. Each treatment contained 16 slices from 4 animals. Similar to the immunohistochemistry results, treatment with 50 μM Aβ increased the levels of phosphorylated tau within the hippocampal slices. The Aβ-induced tau phosphorylation was prevented by pretreatment with 1 nM sAPPα. Electron microscopy demonstrated the presence of long, straight filaments within the cytoplasm of several neurons from hippocampal slices treated with 50 μM Aβ. Many of the filaments were paired, ˜15-29 nm in diameter, and were often observed next to a fragmented nucleus with condensed chromatin. Some paired filaments were twisted with a periodicity of ˜60-120 nm consistent with early tangle formation. These filaments were not observed in slices treated with 50 μM reverse Aβ.

High levels of Aβ induced tau phosphorylation, paired filament formation, and neuronal death in mouse hippocampal slices. Mice overexpressing mutant APP and containing micromolar levels of Aβ, however, do not develop these pathologies. In order to determine if this is due to the expression of neuroprotective polypeptides, a mouse line that overexpresses APP_{Sw} was examined.

### Example 4

### Neuroprotective Genes and Polypeptides are Increased in Aged APP_{Sw} Mice, But Not in AD Patients

In contrast to gene expression levels in AD, the mRNA levels of several growth factors and transthyretin (TTR, an amyloid sequestration polypeptide) are upregulated in 12-month-old APP_{Sw} mice (Table 1). Briefly, the hippocampi from 12-month-old male, non-transgenic (n=2) and APP_{Sw} mice (n=2) were processed for oligonucleotide analysis as described herein. The average fold change was calculated from a 2×2 cross, and significantly changed genes were determined using the Wilcoxon signed rank test for each comparison. Probe sets with p-values<0.001 were called Increased/Decreased; probe sets with p-values in the range 0.001<p-value<0.005 were called Marginally Increased/Decreased; and the remaining probe sets were called No Change. An additional level of ranking was used to incorporate multiple comparisons such that No Change=0, Marginal Increase/Decrease=1/−1, and Increase/Decrease=2/−2. The final rank equaled the sum of the ranks from the 4 comparisons, and the value varied from −8 to 8. The cutoff values for the final determination of increased or decreased gene expression were set as rank≧4 and FC≧1.5 for increased genes and rank≦−4, and FC≦−1.5 for decreased genes.

Insulin-like growth factor 2 is upregulated at both 6 months of age (pre-plaque; Stein and Johinson, J. Neurosci., 22:7380-7388 (2002)) and 12 months of age (post-plaque; Table 1) in APP_{Sw} mice. At 12 months of age insulin is upregulated 11-fold within the hippocampus of APP_{Sw} mice (Table 1). Both IGF-2 and insulin can bind to the IGF-1 receptor and activate a survival pathway that culminates in BAD phosphorylation.

TABLE 1
Differentially expressed genes in 12 month-old APP_{Sw} mice.

| Classification | Gene name | FC | Rank |
|---|---|---|---|
| Amyloid | Transthyretin | 10.05 ± 7.89 | 4 |
| Sequestration | | | |
| Cholesterol | Sterol-C5-desaturase | 1.82 ± 0.11 | 6 |
| | Low density lipoprotein receptor | 1.51 ± 0.07 | 4 |
| Extracellular matrix | Dermatan sulphate proteoglycan 3 | 10.25 ± 4.31 | 6 |
| & tissue | Procollagen, type VIII, alpha 1 | 3.65 ± 1.13 | 4 |
| Remodeling | Decorin | 1.68 ± 0.31 | 4 |
| | Protocadherin alpha 5 | 1.51 ± 0.11 | 6 |
| Fatty acid | Acetyl-Coenzyme A acyltransferase 2 | 1.53 ± 0.23 | 4 |
| Metabolism | (mitochondrial 3-oxoacyl-Coenzyme A | | |
| | thiolase) | | |
| Fibroblast & glial | Glial fibrillary acidic protein | −1.65 ± 0.31 | −4 |
| Growth | Insulin II | 10.95 ± 5.35 | 4 |
| | Phosphatase and tensin homolog (Pten) | 3.12 ± 0.43 | 8 |
| | Prolactin receptor | 3.00 ± 0.82 | 4 |
| | Bone morphogenetic protein 6 | 2.86 ± 0.95 | 4 |
| | Growth hormone | 2.81 ± 0.92 | 4 |
| | Insulin-like growth factor 2 | 2.48 ± 0.76 | 4 |
| | Frizzled homolog 4 | 1.65 ± 0.19 | 4 |
| | Nerve growth factor, beta | −8.79 ± 0.78 | −8 |
| GTPase | Guanylate nucleotide binding protein 2 | 5.80 ± 5.40 | 4 |
| | Guanylate nucleotide binding protein 3 | 2.22 ± 0.63 | 4 |
| | Interferon-inducible GTPase | 5.61 ± 1.56 | 4 |
| | Interferon gamma induced GTPase | 2.16 ± 0.34 | 4 |
| | Retinitis pigmentosa GTPase regulator | 1.82 ± 0.27 | 4 |
| | RAB6, member RAS oncogene family | 1.53 ± 0.14 | 6 |
| | RAP2B, member of RAS oncogene family | 1.51 ± 0.09 | 5 |
| | Guanine nucleotide binding protein, beta 1 | −2.68 ± 0.78 | −5 |
| | cAMP-regulated guanine nucleotide | −1.51 ± 0.21 | −6 |
| | exchange factor II | | |
| Immune-related | T-cell specific GTPase | 6.71 ± 4.05 | 4 |
| | Histocompatibility 2, K region locus 2 | 4.23 ± 1.95 | 4 |
| | Similar to histocompatibility 2, D region | 2.13 ± 0.45 | 4 |
| | locus 1, clone MGC: 25703 | | |
| | Mouse Ia-associated invariant chain (Ii) | 1.71 ± 0.21 | 4 |
| | mRNA fragment | | |
| | Histocompatibility 2, K region | 1.65 ± 0.22 | 5 |
| | Histocompatibility 2, Q region locus 7 | 1.55 ± 0.15 | 6 |
| | Lymphocyte specific formin related protein | −1.73 ± 0.40 | −6 |
| Ion Channels | Voltage-dependent anion channel 2 | 4.02 ± 0.96 | 4 |
| | Potassium voltage gated channel, shaker | 2.52 ± 0.16 | 8 |
| | related subfamily, member 1 | | |
| | ATPase, Na+/K+ transporting, beta 2 | −1.77 ± 0.37 | −6 |
| | polypeptide | | |
| | Chloride channel 4-2 | −1.54 ± 0.12 | −8 |
| Microtubule | Kinesin heavy chain member 5C | −2.35 ± 0.48 | −4 |
| | MAP/microtubule affinity-regulating kinase 2 | −1.50 ± 0.18 | −5 |
| Protease | Cathepsin C | 14.27 ± 7.86 | 5 |
| | Angiotensin converting enzyme | 1.78 ± 1.07 | 4 |
| Protein | Ribosomal protein S11 | 2.16 ± 0.49 | 4 |
| Biosynthesis | Ribosomal protein L29 | 1.79 ± 0.23 | 4 |
| | Eukaryotic translation initiation factor 2C, 2 | 1.66 ± 0.17 | 8 |
| Proteosome | Proteosome (prosome, macropain) subunit, | 5.70 ± 2.71 | 4 |
| | beta type 9 (large multifunctional protease 2) | | |
| | Proteosome (prosome, macropain) subunit, | 1.82 ± 0.35 | 4 |
| | beta type 8 (large multifunctional protease 7) | | |
| Synaptic | Vesicle-associated membrane protein 3 | −1.88 ± 0.24 | −6 |
| | Synaptobrevin 2 | −1.86 ± 0.29 | −8 |
| Thyroid hormone | Peroxisome proliferator-activated receptor | 2.59 ± 0.32 | 8 |
| | binding protein | | |
| | Thyroid hormone receptor alpha | −1.59 ± 0.12 | −5 |
| Transcription | Trans-acting transcription factor 4 | 8.75 ± 5.29 | 5 |
| Regulation | Chromobox homolog 3 (Drosophila | 2.71 ± 0.37 | 8 |
| | HP1 gamma) | | |
| | Trans-acting transcription factor 1 | 2.70 ± 0.61 | 8 |
| | Early growth response 1 | 2.20 ± 0.39 | 4 |
| | Sox11 SRY-box containing gene 11 | 2.03 ± 0.16 | 8 |
| | Nuclear receptor interacting protein 1 | 1.67 ± 0.19 | 6 |
| | Myocyte enhancer factor 2C | 1.60 ± 0.19 | 6 |
| | Transcription factor 20 | 1.51 ± 0.12 | 4 |
| Miscellaneous | Retinol dehydrogenase type 5 | 6.34 ± 3.13 | 4 |
| | Aquaporin 1 | 5.48 ± 2.60 | 4 |
| | Folate receptor 1 (adult) | 4.25 ± 2.77 | 4 |
| | LPS-binding protein | 2.59 ± 0.76 | 4 |
| | Cyclin-dependent kinase inhibitor 1C (P57) | 2.41 ± 0.73 | 4 |
| | DNA segment, Chr 7, Roswell Park 2 | 2.21 ± 0.59 | 6 |
| | Complex, expressed | | |
| | DEAD (aspartate-glutamate-alanine-aspartate) | 2.08 ± 0.15 | 8 |
| | box polypeptide 6 | | |
| | Kidney cell line derived transcript 1 | 2.05 ± 0.58 | 4 |
| | Paternally expressed 3 | 1.99 ± 0.18 | 8 |
| | Ganglioside-induced differentiation | 1.97 ± 0.19 | 8 |
| | associated protein 10 | | |
| | Prosaposin | 1.89 ± 0.23 | 8 |
| | Splicing factor 3b, subunit 1, 155 kDa | 1.79 ± 0.05 | 8 |
| | Close homolog of L1 | 1.73 ± 0.09 | 8 |
| | Farnesyl diphosphate synthetase | 1.72 ± 0.11 | 8 |
| | Solute carrier family 31, member 1 | 1.67 ± 0.27 | 4 |
| | DNA segment, Chr X, ERATO Doi 242, | 1.64 ± 0.15 | 6 |
| | expressed | | |
| | Cofilin 2, muscle | 1.64 ± 0.16 | 4 |
| | Ankyrin 3, epithelial | 1.63 ± 0.11 | 8 |
| | Phosphodiesterase 1A, calmodulin-dependent | 1.62 ± 0.04 | 8 |
| | B-cell receptor-associated protein 37 | 1.61 ± 0.16 | 4 |
| | NMDA receptor-regulated gene 1 | 1.53 ± 0.10 | 6 |
| | DNA segment, Chr 9, Wayne State | 1.53 ± 0.07 | 4 |
| | University 18, expressed | | |
| | SWI/SNF related, matrix associated, actin | 1.50 ± 0.08 | 4 |
| | dependent regulator of chromatin, | | |
| | subfamily c, member 1 | | |
| | Farnesyltransferase, CAAX box, beta | −6.62 ± 3.75 | −4 |
| | Solute carrier family 1, member 1 | −4.39 ± 1.89 | −4 |
| | Phosphatidylinositol-4-phosphate 5-kinase, | −2.80 ± 0.49 | −4 |
| | type 1 gamma | | |
| | Karyopherin (importin) alpha 6 | −2.49 ± 0.78 | −4 |
| | Nel-like 2 homolog (chicken) | −1.64 ± 0.29 | −6 |
| | Cytokine receptor-like factor 1 | −1.61 ± 0.18 | −7 |
| | Dentatorubral pallidoluysian atrophy | −1.60 ± 0.24 | −6 |
| | Adrenergic receptor kinase, beta 1 | −1.55 ± 0.23 | −6 |
| | Neurogranin (protein kinase C substrate, RC3) | −1.53 ± 0.14 | −7 |
| | Latent transforming growth factor beta | −1.53 ± 0.17 | −8 |
| | binding protein 4 | | |
| | Metallothionein 3 | −1.52 ± 0.26 | −4 |
| | Non-POU-domain-containing, octamer | −1.52 ± 0.03 | −4 |
| | binding protein | | |
| ESTs | R75193 | 3.07 ± 0.18 | 8 |
| | AV281129 | 2.10 ± 0.35 | 4 |
| | AI561567 | 2.06 ± 0.14 | 8 |
| | AW123795 | 2.05 ± 0.11 | 8 |
| | C79052 | 2.05 ± 0.12 | 5 |
| | AA623874 | 2.04 ± 0.28 | 4 |
| | AA794350 | 1.97 ± 0.25 | 5 |
| | AW123567 | 1.95 ± 0.28 | 6 |
| | AA717225 | 1.95 ± 0.28 | 5 |
| | AI551087 | 1.83 ± 0.20 | 7 |
| | AV319920 | 1.82 ± 0.28 | 6 |
| | AA874329 | 1.81 ± 0.11 | 8 |
| | AW046672 | 1.71 ± 0.12 | 4 |
| | AW121960 | 1.70 ± 0.17 | 6 |
| | AW108350 | 1.69 ± 0.17 | 6 |
| | AA688938 | 1.68 ± 0.25 | 4 |
| | AI851770 | 1.67 ± 0.02 | 7 |
| | AV349142 | 1.65 ± 0.11 | 5 |
| | AI642098 | 1.59 ± 0.30 | 4 |
| | AI843258 | 1.57 ± 0.03 | 7 |
| | AW046038 | 1.57 ± 0.33 | 4 |
| | AA759910 | 1.54 ± 0.17 | 7 |
| | AA692708 | 1.52 ± 0.08 | 4 |
| | AW049142 | −2.14 ± 0.19 | −5 |
| | AI852916 | −1.81 ± 0.26 | −4 |
| | AW048549 | −1.74 ± 0.19 | −8 |
| | AW120971 | −1.70 ± 0.11 | −4 |
| | AW125433 | −1.70 ± 0.15 | −6 |
| | M26005 | −1.70 ± 0.26 | −4 |
| | AW048484 | −1.56 ± 0.14 | −8 |
| | AI851703 | −1.56 ± 0.18 | −6 |

Rank is based on the p-value for each comparison (2 × 2) such that a rank of 8/−8 corresponds to a p-value <0.001.
Rank values ranging from 4 to 8 indicate significantly increased gene expression (Roman text), and values from −4 to −8 indicate significantly decreased genes (italic text).
FC, fold change.
FC is expressed as mean ± SEM.

At the protein level, 12-month-old APP_{Sw} mice possessed dramatically increased levels of TTR and IGF-2 within the extracellular space of the hippocampus and particularly around the neuronal fields. In addition, IGF-2 is localized around some neurons and neuronal processes. BAD is a pro-apoptotic polypeptide in its non-phosphorylated state. However, when phosphorylated at serine 112 or 136, BAD polypeptides bind to 14-3-3 proteins and release the anti-apoptotic BCL-2 family members, BCL-2 and BCL-X_{L}. Immunohistochemistry for phospho-BAD(112) revealed an increase within the NeuN positive neurons of the hippocampal neuronal fields when compared to non-transgenic controls. In contrast, the levels of total BAD within the hippocampal neurons were unchanged between nontransgenic and APP_{Sw} mice.

In AD, many of these changes do not occur and, in fact, may be reversed. For example, the level of total BAD can be increased in AD temporal cortex (Kitamura et al., Brain Res., 780:260-269 (1998)). On the other hand, immunohistochemistry revealed unchanged levels of total BAD between control (n=5) and AD (n=6) patients within the pyramidal neurons of the hippocampus. IGF-2 levels were detectable around the pyramidal neurons of the hippocampal neuronal fields and were not consistently different between control and AD patients. TTR levels have been demonstrated to be reduced in the CSF of AD patients (Serot et al., J. Neurol. Neurosurg. Psychiatry, 63:506-508 (1997)). Little to no TTR was observed in or around the hippocampal neuronal fields of control or AD patients as detected by immunohistochemistry of post-mortem brains. TTR deposits, however, co-localized with many Aβ plaques within the hippocampus of all AD patients. Neither IGF-2 nor a control goat IgG were observed to stain Aβ plaques in the AD patients. In addition, occasional neurons within the hippocampus of AD patients contained high levels of intracellular Aβ that co-localized with TTR. Vertical sections revealed definite co-localization of TTR with Aβ. These results are representative of those obtained from 5 controls (mean age=71.4±2.9 yr) and 6 AD patients (mean age=77.3±3.8 yr).

### Example 5

### sAPPα-driven Gene and Polypeptide Expression

One major difference between the mouse models and human AD is the overexpression of full-length APP, which may be 5-fold or more in the mouse (Hsiao et al., Science, 274:99-102 (1996)). Thus, unlike in AD, all the cleavage products of APP likely are upregulated in the mouse models overexpressing mutant APP. As demonstrated herein, sAPPα can protect against Aβ-induced tau phosphorylation and neuronal death. Thus, high levels of sAPPα in the APP_{Sw} mice might be responsible for the increased levels of the neuroprotective TTR, IGF-2, and phospho-BAD.

Briefly, dissected hippocampi from 4 APP_{Sw} mice and 4 nontransgenic controls were homogenized separately in lysis buffer (50 mM Tris, 150 mM NaCl, 1 mM EDTA, 1% NP40, 1 M PMSF, 3 mM benzamidine, 200 μM leupeptin, 20 μM aprotinin, 100 μM sodium orthovanadate, 2 mM dithiothreitol). After centrifugation at 12000×g for 10 minutes, the soluble fraction was taken. Total protein levels were determined using the bicinchoninic acid (BCA) colorimetric assay (Pierce, Rockford, Ill.). SDS-reducing buffer (50 mM Tris, 10% glycerol, 2% SDS, 0.1% bromphenol blue, pH 6.8) was added to the tissue lysates, and the samples were heated to 95° C. for 10 minutes. 50 μg of total protein per well was loaded and separated on a 7.5% SDS-PAGE gel. The gel was transferred to a polyvinylidene difluoride membrane, and the membrane was immunoblotted with a 1:200 dilution of6E10, a monoclonal antibody against sAPPα (Chemicon). Bands were visualized using a horseradish peroxidase-conjugated anti-mouse IgG antibody (1:2000) and the SuperSignal West Pico chemiluminescent substrate (Pierce). Bands were quantified by using the Molecular Dynamics 300 A Computing Densitometer (Sunnyvale, Calif.). In order to estimate hippocampal sAPPα concentrations, a standard curve was run on sAPPα, and an estimated mouse hippocampal volume of 23 μL was used (based on hippocampal weights).

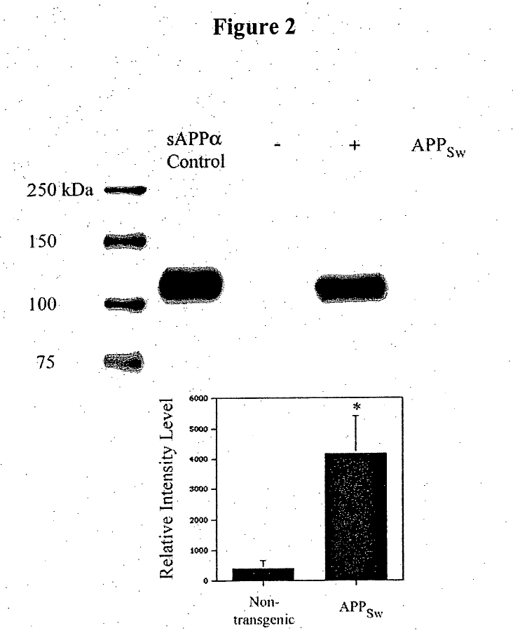

Non-transgenic mice were found to have virtually undetectable levels of sAPPα in the hippocampus, while APP_{Sw} mice possessed significantly higher amounts (FIG. 2). The 4G8 antibody binds to full-length APP and Aβ, but not to sAPPα, and does not recognize the band shown in FIG. 2. However, a weak band at ˜130 kDa that represents full-length APP was recognized by 6E10 and 4G8 antibodies in the APP_{Sw} mice. This band was weak compared to the band for sAPPα, indicating that the majority of APP is cleaved by α- or β-secretase. Using immunoblotting, the average concentration of sAPPα in the APP_{Sw} mouse hippocampus was determined to be 1.2±0.7 μM.

Next, organotypic hippocampal slices were treated with vehicle or 1 nM sAPPα for 24 hours; the RNA was isolated; and oligonucleotide microarray analysis was performed. Samples consisted of slices treated with vehicle (n=3) or sAPPα (n=3). Each sample pooled 8-12 slices from 4 animals. The average fold change was calculated from a 3×3 comparison. Treatment with sAPPα resulted in a significant increase (rank ≧9) in the expression levels of 45 genes and ESTs (Table 2). No genes or ESTs were significantly decreased by sAPPα treatment. Similar to the adult APP_{Sw} mice, ttr was one of the genes with the greatest fold change (8.9-fold). In addition, the mRNA levels of igf-2 and insulin-like growth factor binding protein 2 (igfbp2) were increased by sAPPα. Several other genes involved in protective pathways such as apoptosis inhibition, detoxification, and retinol transport were upregulated by sAPPα (Table 2).

TABLE 2
Differentially expressed genes in sAPPα-treated organotypic hippocampal slice
cultures.

| Classification | Gene Name | FC | Rank |
|---|---|---|---|
| Amyloid | Transthyretin | 8.94 ± 4.49 | 12 |
| Sequestration | | | |
| Apoptosis | Apoptosis inhibitor 6 | 2.09 ± 1.12 | 10 |
| Calcium binding | Calmodulin-like 4 | 1.75 ± 0.42 | 12 |
| Cell Cycle | Cyclin D2 | 4.22 ± 0.88 | 11 |
| | Cyclin-dependent kinase 4 | 1.26 ± 0.05 | 10 |
| Detoxification | Glutathione S-transferase, alpha 4 | 3.52 ± 1.11 | 14 |
| | Glutathione S-transferase, mu 5 | 1.38 ± 0.40 | 9 |
| | Peroxiredoxin 2 | 1.19 ± 0.31 | 10 |
| Extracellular matrix | Elastin | 1.66 ± 1.22 | 9 |
| & tissue remodeling | Chondroitin sulfate proteoglycan 2 | 1.62 ± 0.21 | 11 |
| Glycolysis | Phosphofructokinase, platelet | 1.32 ± 0.06 | 12 |
| | Galactokinase 1 | 1.28 ± 0.30 | 10 |
| Growth | Insulin-like growth factor 2 | 2.58 ± 0.42 | 14 |
| | Insulin-like growth factor binding | 1.53 ± 0.08 | 14 |
| | protein 2 | | |
| Immune-related | Lymphocyte antigen 6 complex, locus A | 1.90 ± 0.97 | 12 |
| | Intercellular adhesion molecule 2 | 1.71 ± 0.73 | 9 |
| Ion Channel | FXYD domain-containing ion | 1.20 ± 0.30 | 10 |
| | transport regulator 1 | | |
| Peroxisome (Fatty | ATP-binding cassette, sub-family D | 1.35 ± 0.12 | 9 |
| acid transport) | (ALD), member 3 | | |
| Prostaglandin | Prostaglandin D2 synthase (brain) | 1.97 ± 0.53 | 12 |
| Synthesis | | | |
| Protein catabolism | Proteasome (prosome, macropain) | 2.67 ± 0.73 | 10 |
| | 28 subunit, alpha | | |
| | Praja1, RING-H2 motif containing | 1.20 ± 0.03 | 9 |
| Receptor | Cytokine receptor-like factor 1 | 1.54 ± 0.45 | 9 |
| | Receptor (calcitonin) activity | 1.27 ± 0.06 | 9 |
| | Modifying protein 1 | | |
| Retionic acid | Cellular retinoic acid binding | 2.37 ± 0.29 | 16 |
| Binding | protein II | | |
| | Retinol binding protein 1, cellular | 1.69 ± 0.15 | 16 |
| Miscellaneous | Eyes absent 4 homolog (Drosophila) | 19.26 ± 8.88 | 11 |
| | H19 fetal liver mRNA | 1.95 ± 0.23 | 17 |
| | Ectonucleotide | 1.88 ± 0.82 | 11 |
| | pyrophosphatase/phosphodiesterase 2 | | |
| | Phosphatidic acid phosphatase 2a | 1.83 ± 0.78 | 10 |
| | Heterogeneous nuclear | 1.34 ± 0.35 | 10 |
| | ribonucleoproteins methyltransferase- | | |
| | like 2 (S. cerevisiae) | | |
| | Topoisomerase (DNA) II alpha | 1.34 ± 0.96 | 10 |
| | Neuronal protein 15.6 | 1.33 ± 0.05 | 10 |
| | Ribosomal protein L8 | 1.32 ± 0.08 | 11 |
| | Ena-vasodilator stimulated | 1.28 ± 0.35 | 10 |
| | phosphoprotein | | |
| | Thymus cell antigen 1, theta | 1.27 ± 0.32 | 11 |
| | ADP-ribosylation-like factor 6 | 1.25 ± 0.04 | 14 |
| | Interacting protein 5 | | |
| | STIP1 homology and U-Box | 1.19 ± 0.05 | 9 |
| | Containing protein 1 | | |
| ESTs | AW060684 | 4.54 ± 1.78 | 9 |
| | AW121164 | 2.54 ± 0.86 | 9 |
| | AW121336 | 2.50 ± 0.34 | 9 |
| | AW060956 | 1.35 ± 0.11 | 9 |
| | AW120814 | 1.32 ± 0.11 | 9 |
| | AW124069 | 1.26 ± 0.34 | 9 |
| | AW048976 | 1.24 ± 0.08 | 10 |
| | AI226264 | 1.21 ± 0.03 | 10 |

Rank is based on the p-value for each comparison (3 × 3) such that a rank of 18 corresponds to a p-value <0.01.
Rank values ranging from 9 to 18 indicate significantly increased gene expression.
FC, fold change.
FC is expressed as mean ± SEM.

Immunohistochemistry revealed a dramatic increase in both TTR and IGF-2 within the extracellular space of 1 nM sAPPα-treated hippocampal slices. Polypeptide levels appeared highest around the hippocampal neuronal fields. Consistent with the increase in IGF-2 and similar to adult APP_{Sw} mice, levels of phospho-BAD were increased within the NeuN positive neurons of sAPPα-treated hippocampal slices.

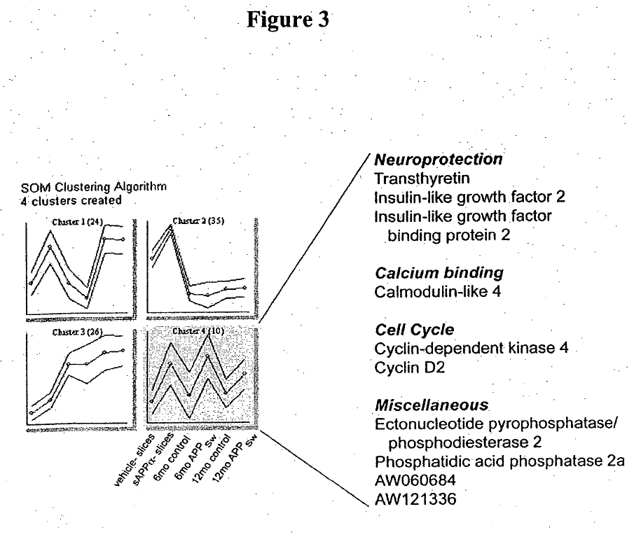

The relative expression levels of the genes increased by sAPPα treatment in the hippocampal slice cultures were clustered together with the expression profiles of 6-month-old and 12-month-old nontransgenic and APP_{Sw} mice. Self-organized map (SOM) clustering generated 4 clusters. Cluster 4 (FIG. 3, lower right panel) includes those genes and ESTs with low expression levels in vehicle-treated slices, 6-month-old control mice, and 12-month-old control mice and high expression levels in sAPPα-treated slices, 6-month-old APP_{Sw} mice, and 12-month-old APP_{Sw} mice. Therefore, this is a cluster of genes that is increased by sAPPα ex vivo and, likely, in vivo. Genes in this cluster include ttr, igf-2, and igfbp2 (FIG. 3).

### Example 6

### TTR and IGF-2 Participate in sAPPα-induced Protection Against Aβ Toxicity

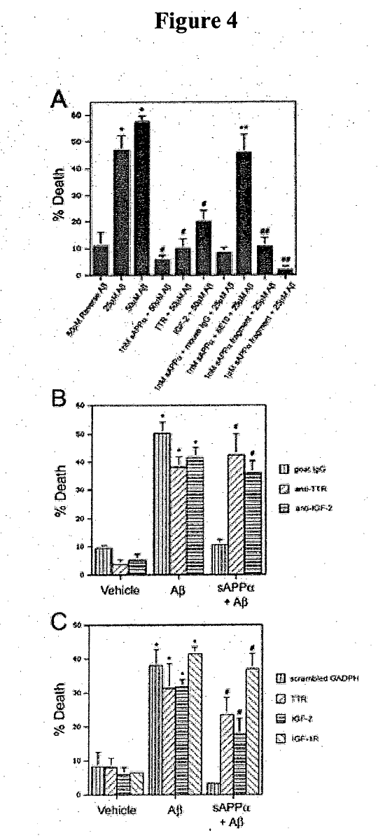

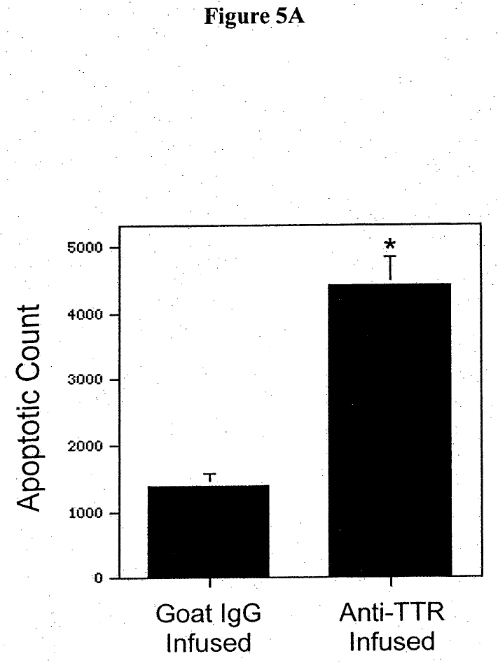

Organotypic hippocampal cultures were treated and incubated with ethidium homodimer (EthD-1) and calcein AM. The Aβ-induced EthD-1 staining occurred within the NeuN positive neurons of the hippocampal neuronal fields. Here, the numbers of cells stained with each fluorescent probe were counted and expressed as the percent of cells that are EthD-1 positive and thus have lost membrane integrity (% Death; FIG. 4). Treatment with either 25 μM or 50 μM Aβ resulted in a dramatic increase in the percent death compared to 50 μM reverse Aβ treatment. Pretreatment with 1 nM sAPPα for 48 hours completely protected against Aβ-induced death. When added together with Aβ, 3 μM TTR also completely protected against Aβ, while exogenous addition of 500 nM IGF-2 partially protected against Aβ (FIG. 4A). Further, addition of an antibody that recognizes the COOH-terminal 17 amino acids of sAPPα (antibody 6E10) prevented the protective effect of sAPPα. This region contains part of the Aβ sequence and is unique to APP cleaved by α-secretase versus β-secretase. In addition, treatment with a 10 amino acid fragment that corresponds to part of this sequence mimics the protective effect of sAPPα (FIG. 4A).

Because both TTR and IGF-2 are secreted proteins, it is possible to interfere with their function by adding antibodies raised against them. Addition of a control goat IgG together with sAPPα did not interfere with sAPPα-induced protection against Aβ. However, addition of antibodies directed against TTR or IGF-2 prevented the protection by sAPPα (FIG. 4B). Supporting this, siRNA knock-down of TTR or IGF-2 partially prevented the sAPPα-induced protection against Aβ (FIG. 4C). IGF-2 may protect cells by activating the IGF-1 receptor and causing the phosphorylation of BAD. For example, siRNA knock-down of IGF-1R also prevented protection by sAPPα (FIG. 4C). All knock-downs were confirmed by immunohistochemistry, and siRNA of either IGF-2 or IGF-1R prevented the BAD phosphorylation induced by sAPPα. The fact that the inhibition or knock-down of either TTR polypeptides or IGF-2 polypeptides significantly blocked protection suggests that induction of both TTR polypeptides and IGF-2 polypeptides are involved for maximum protection against Aβ.

### Example 7

### TTR Protects APP_{Sw} Mice From Neurodegeneration

An antibody to TTR was directly infusing into the CA1 region of the APP_{Sw} mouse hippocampus in an attempt to prevent the sequestration of Aβ. Osmotic pumps were used to perform a continuous infusion of the anti-TTR antibody over a 2 week period. This resulted in a dramatic deposition of the anti-TTR antibody within the extracellular space of the infused hippocampus, but not the non-infused hippocampus. Infusion of a control goat IgG did not result in goat IgG deposition. Moreover, APP_{Sw} mice infused with the anti-TTR antibody demonstrated markedly increased levels of Aβ in and around the hippocampal neuronal fields when compared to goat IgG or the non-infused hippocampus. This suggests that the anti-TTR antibody disrupted the TTR polypeptide binding of Aβ and resulted in a locally increased Aβ load. Many plaques within the anti-TTR infused hippocampus co-stained with Aβ and the anti-TTR antibody, suggesting that TTR polypeptides bind to the Aβ plaques in vivo in the APP_{Sw} mice. Non-transgenic mice did not produce or possess any detectable TTR polypeptide within their hippocampi. In fact, infusion of the anti-TTR antibody into non-transgenic mice did not result in accumulation of either the antibody or Aβ, and no tau phosphorylation could be detected.

In addition to causing Aβ accumulation around the CA1 neuronal field, infusion of the anti-TTR antibody led to tau phosphorylation within many CA1 neurons. Several neurons immunopositive with the AT8 antibody were present within the CA1 field of the anti-TTR infused hippocampus while no cells stained positively in goat IgG infused or non-infused hippocampi. The AT8 positive neurons often contained pyknotic nuclei indicative of degeneration. An antibody that recognizes tau phosphorylated at Thr231 also stained CA1 neurons in the anti-TTR infused hippocampus. These phosphorylated tau positive neurons co-stained with the neuronal marker NeuN. The number of phospho-tau(Thr231) positive cells and the intensity of staining were not altered in goat IgG and non-infused hippocampi.

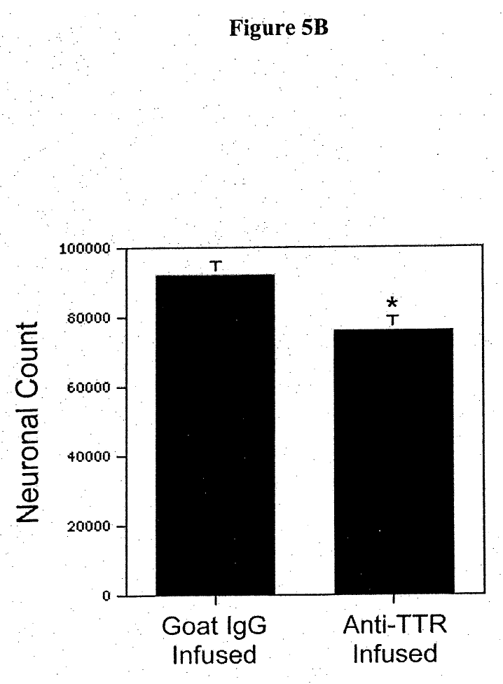

In order to determine if the number of apoptotic cells was increased and the total number of healthy neurons decreased in CA1 following the anti-TTR antibody infusion, unbiased stereology using the optical fractionator was performed. Apoptotic neurons are cleared within 72 hours in the in vivo nervous system (Hu et al., J. Neurosci., 17:3981-3989 (1997)). Despite this, after 2 weeks of continuous infusion with goat IgG some cells with pyknotic nuclei were counted within the CA1 pyramidal neuronal field. Infusion of the anti-TTR antibody significantly increased the total number of cells with pyknotic nuclei in CA1 (Table 3 and FIG. 5A). As a control, no pyknotic cells could be observed in a control mouse without an infusion. Consistent with increased neuronal apoptosis, the total number of CA1 pyramidal neurons was significantly decreased by 17.1±5.3% in mice infused with the anti-TTR antibody compared to mice infused with goat IgG (Table 2 and FIG. 5B). Table 3. Estimated total number of cells with pyknotic nuclei and neurons with healthy nuclei within CA1 of each APP_{Sw} mouse infused with either goat IgG or the anti-TTR antibody.

| | Pyknotic cell count | | Neuronal count | |
|---|---|---|---|---|
| Infusion | goat IgG | anti-TTR | goat IgG | anti-TTR |
| | 1782 | 3289 | 88400 | 83880 |
| | 1486 | 5413 | 89260 | 81940 |
| | 955 | 4352 | 103590 | 70260 |
| | 1380 | 4564 | 87780 | 69840 |
| Mean ± | 1401 ± 171 | 4405 ± 437 | 92260 ± 3790 | 76480 ± 3730 |
| SEM | | | | |

In summary, AD may be caused by the abnormal processing of the amyloid precursor protein (APP) and the accumulation of β-amyloid (Aβ). However, mice overexpressing mutant APP do not develop the tau phosphorylation or neuronal loss characteristic of the human disease. As described herein, an ex vivo model of the mouse hippocampus was developed such that Aβ treatment leads to the phosphorylation of tau and neuronal death. In contrast, osecretase cleaved APP (sAPPα) protects against these Aβ-induced pathologies and increases the expression levels of several neuroprotective genes. The sAPPα-driven expression of transthyretin and insulin-like growth factor 2 is involved in the protection against Aβ-induced neuronal death in organotypic hippocampal cultures. Chronic infusion of an antibody against transthyretin into the hippocampus of mice overexpressing APP_{Sw} leads to increased Aβ, tau phosphorylation, and neuronal loss and apoptosis within the CA1 neuronal field. Therefore, the elevated expression of transthyretin is mediated by sAPPα and protects APP_{Sw} mice from developing many of the neuropathologies observed in AD. This model system more closely represents the architecture and chemistry of the intact brain and revealed that aggregated Aβ can induce some of the major pathological features of AD. In addition, the slice cultures provided herein can allow scientists to explore the precise mechanisms by which Aβ leads to the phosphorylation of endogenous tau and neuronal death.

Again, Aβ is capable of inducing tau phosphorylation and apoptosis in an ex vivo model of the mouse hippocampus. These pathologies also are observed in vivo when an antibody directed against the Aβ-binding protein TTR is infused in APP_{Sw} mice. The results presented herein demonstrate that sAPPα replacement or activation of sAPPα-induced pathways may help prevent the toxicity of Aβ and the development of AD.

### Example 8

### Neuropathology and Protection Induced in Organotypic Cortical Cultures From Adult Humans

Subjects

Four patients with intractable epilepsy who had elected to undergo a temporal lobe resection were recruited for this study. These patients often possess a hippocampus with sclerosis. However, the overlying temporal cortex is normal. The age of the subjects ranged from 20 to 49. Brain tissue was obtained with informed consent from all patients.

Human Organotypic Cultures

Brain tissue was removed in large, intact pieces by electrocautery. Tissue obtained from the temporal cortex was approximately 3 cm×2 cm×1.5-2 cm. Immediately following removal, the tissue was placed in ice-cold Neuregen-I media (Brainbits, Springfield, Ill.), a CO₂ independent transport and holding medium, and transported to a tissue culture laboratory. Meninges were removed before cutting the tissue with a scalpel to produce blocks about 0.5-1 cm³ such that each gyrus was kept as intact as possible and the cortical layers were preserved. As described previously, incisions were made perpendicular to the longitudinal axis of the gyrus and along the white matter (Verwer et al., Exp. Gerontol., 38:167-172 (2003)). A vibratome was used to cut 400 μm thick slices at a plane perpendicular to the longitudinal axis of the gyrus. Thus, all cortical layers and a small amount of white matter were included in a slice. Alternately, small tissue pieces, which were usually obtained from the hippocampus, were cut at 400 μm using a Mcllwain tissue chopper. No more than 2 slices were placed on a filter insert with a 30 mm diameter and a 0.4 μm filter pore size (Millipore, Billerica, Mass.) held in 6-well culture plates. For the first 12 hours, slices were kept in 1-1.2 mL Neurobasal media with B-27 supplement, 0.5 mM glutamine, and 1% penicillin/streptomycin (Invitrogen). Subsequently, the media was changed every 2 days using 1 mL of media without antibiotics. Organotypic cultures were maintained in a humidified incubator at 37° C. in 5% O₂, 5% CO₂, and 90% N for 4 to 28 days.

Cell death and viability was determined on live slices using the fluorescent probes from Molecular Probes' Live/Dead Kit. After a 40 minute incubation in calcein AM (1:400) and ethidium homodimer (1:1000) in phosphate buffered saline, slices were visualized at 200× magnification with an inverted Nikon Diaphot 200 microscope using the Bio-Rad MRC-1024 Laser Scanning Confocal system (Hercules, Calif.). Random images were captured at the emission spectrum of each probe, and Scion Image software (Frederick, Md.) was used to quantify the number of live and dead cells. At least three areas were imaged and used to determine the percent death for each slice. The percent death was calculated as the number of EthD-1 positive cells divided by the total number of EthD-1 and calcein AM positive cells.

Treatments

β-amyloid₁₋₄₂ and reverse Aβ (A₄₂₋₁) were obtained from BACHEM (Torrance, Calif.). These peptides were dissolved in 0.1% NH₃OH at 222 μM. For treatments, Aβ or reverse Aβ was aggregated at 50 μM in Neurobasal media with B-27 supplement and 0.5 mM glutamine at 37° C. for 24 hours. Then, the slice culture media was removed, and the slices were treated with 1 mL of 25 μM or 50 μM Aβ, reverse Aβ, or vehicle for 24 hour. Human TTR (Calbiochem) was added to slices together with the Aβ treatment at final concentrations of 0.03, 0.3, and 3 μM TTR. sAPPα was obtained from Sigma (St. Louis, Mo.). Unless otherwise stated, all sAPPα treatments were performed at 1 nM 48 hours prior to the addition of Aβ. The 10 amino acid fragment of the carboxyl-terminal region of sAPPα (592-601 of APP695; SEVKMDAEFR was added according to the same protocol for sAPPα. Because a variable amount of tissue was obtained from each patient, not all treatments were performed on all patients.

Detection of Cell Death by TUNEL Staining

Cortical slice cryosections were stained with terminal deoxynucleotidyl transferase-mediated, dUTP nick end labeling (TUNEL) and with an anti-NeuN antibody. Cells with DNA fragmentation were determined by the terminal deoxynucleotidyl transferase incorporation of fluorescein isothiocyanate (FITC)-12-dUTP into DNA (In Situ Cell Death Detection kit, Roche Biochem, Indianapolis, Ind.). At least 3 random images per slice were generated by confocal analysis at 600× magnification. The number of TUNEL positive, NeuN positive, and both TUNEL and NeuN positive cells were then quantified.

Immunohistochemistry

Slices were fixed in 4% PFA for 20 minutes, incubated in 30% sucrose in phosphate buffered saline overnight, and frozen in OCT embedding medium. Frozen sections with a width of 10 μm were taken through each slice. Phosphorylated tau was detected with the monoclonal AT8 antibody (1:200; Research Diagnostics, Flanders, N.J.) and anti-phospho-tau(Thr231) (1:500; Calbiochem, San Diego, Calif.). As a control, pre-immune rabbit or mouse IgG (Vector Laboratories, Burlingame, Calif.) was used in place of the primary antibody. A biotinylated anti-rabbit IgG or anti-mouse IgG was used as the secondary antibody. The Vectastain Elite ABC kit (Vector) and tyramide conjugated to either Alexa Fluor 488 or 568 (Molecular Probes, Eugene, Oreg.) were used to visualize the antibody staining. Anti-NeuN (1:250; Chemicon International, Temecula, Calif.) together with an anti-mouse IgG secondary antibody conjugated to Alexa Fluor 647 was used to detect the neuronal marker NeuN. Sections were imaged using either the Bio-Rad Laser Scanning Confocal system or epifluorescence (Zeiss, Thornwood, N.Y.).

Statistical Analysis

Results are expressed as mean±SEM. Statistical significance was determined using a two-tailed, unpaired student's t test, and a p-value<0.05 was considered significant.

Results

Slices from the temporal cortex of adult humans were maintained in culture for 4 to 21 days. Ethidium homodimer (EthD-1) is a membrane impermeable DNA-binding dye that is excluded from live cells with an intact plasma membrane. Calcein AM is a cell-permeant dye that fluoresces in live cells with a functional intracellular esterase. During the first 3 days in culture, numerous EthD-1 positive cells were observed in control slices. These are likely cells that have been damaged during surgery or the subsequent slicing. By day 4, the percent of EthD-1 positive cells dropped to an average level of 20%. Numerous calcein AM positive cells were present through 21 days in culture. The majority of the calcein AM cells were approximately 20 μm in diameter. In addition, many cells stained positively for the neuronal marker NeuN, indicating the presence of large cortical neurons.

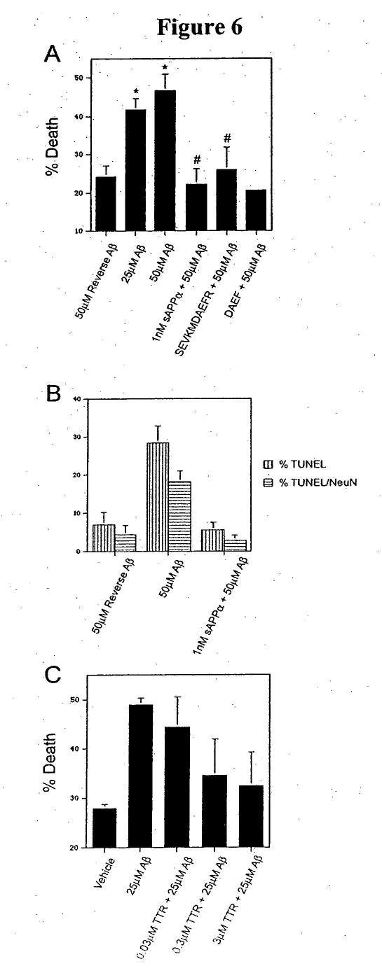

Slices were treated with aggregated Aβ in an attempted to model AD in the slices. Treatment with 50 μM Aβ resulted in a dramatic increase in the number of cells that have lost membrane integrity. Pretreatment with 1 nM sAPPα for 48 hours prevented the Aβ-induced EthD-1 staining. Treatment with 50 μM Aβ significantly increased the percent death, while pretreatment with 1 nM sAPPα significantly protected against the Aβ-induced death (FIG. 6A). A 10 amino acid fragment corresponding to the COOH-terminal region of sAPPα also demonstrated significant protection in four subjects (p-value<0.05; FIG. 6A). In addition, a sample from one subject was treated with a 4 amino acid fragment (DAEF; SEQ ID NO:2). This fragment protected against 50 μM Aβ toxicity (FIG. 6A).

Treatment with 50 μM Aβ also resulted in DNA strand breaks as indicated by TUNEL. Many of these TUNEL positive cells co-labeled with NeuN. Pretreatment with 1 nM sAPPα prevented the Aβ-induced increase in TUNEL positive neurons.

The percent of total NeuN and TUNEL positive cells that stained for TUNEL (% TUNEL) was quantified for 1 subject. In addition, the percent of total NeuN positive cells that co-stained with NeuN and TUNEL was determined (FIG. 6B). Both the percent of total TUNEL positive cells and the percent of neurons that were TUNEL positive were increased following treatment with Aβ (FIG. 6B).

TTR polypeptide expression is induced by sAPPα and is involved in protection against Aβ toxicity. Co-treatment of the human cortical slices with 0.3 or 3 μM TTR polypeptide, but not 0.03 μM TTR polypeptide, protected against the Aβ-induced EthD-1 staining (FIG. 6C).

The monoclonal antibody AT8 recognizes tau phosphorylated at Ser202 and is commonly used to stain early and mature tangles in AD. Cortical slices treated with 50 μM reverse Aβ did not possess any AT8 positive cells. Treatment with 50 μM Aβ, however, resulted in several AT8 positive cells with a somatodendritic redistribution characteristic of early tau pathology. Slices treated with 50 μM reverse Aβ demonstrated some tau phosphorylated at Thr231 within processes, but not in the cell body, of NeuN positive cells consistent with the normal distribution of tau. In contrast, treatment with 50 μM Aβ resulted in several NeuN positive neurons with high levels of somatic phospho-tau(Thr231). Pretreatment with 1 nM sAPPα prevented the Aβ-induced tau phosphorylation. In addition, electron microscopy of slices treated with 50 μM Aβ for 1 week revealed a small number of neurons with tangle-like structures found in the dendrites.

In summary, these results demonstrate that cortical tissue removed from epilepsy patients can be maintained as organotypic slice cultures for up to 21 days, and that Aβ treatment of human cortex results in a loss of cell membrane integrity, neuronal DNA fragmentation, and tau phosphorylation. Aβ induced tau phosphorylation and redistribution to the somatodendritic compartment of neurons where tangle-like structures can be identified by electron microscopy. Pretreatment with sAPPα prevented these neurodegenerative changes. This protection is mimicked by the 10 amino acid sAPPα fragment. In addition, TTR polypeptide itself can protect against Aβ toxicity at concentrations at or above the 0.3 μM of TTR found in normal human CSF (Schwarzman et al., Proc. Natl. Acad. Sci., 91:8368-8372 (1994)). These results demonstrate that increasing the amount of sAPPα, a fragment of sAPPα, and/or a TTR polypeptide in the brain can delay or prevent the onset of AD. These results also demonstrate that Aβ can induce the neurodegeneration associated with AD in human slice cultures. The ex vivo model developed is of particular relevance to AD as compared to other models in that the tissue is: (a) human instead of animal, (b) adult instead of embryonic, fetal, or early postnatal, (c) intact instead of dissociated, and (d) genetically normal instead of transgenic, immortalized, or neoplastic.

### Example 9

### Polypeptide Protection in Mouse Hippocampal Slice Cultures

Hippocampal Organotypic Cultures

Pups from C57B6/SJL mice were anaesthetized and decapitated at postnatal day 15. The brain was removed and placed in ice-cold dissecting medium (50% Minimum Essential Medium (Invitrogen, Carlsbad, Calif.), 50% Hank's Balanced Salt Solution (Invitrogen), 25 mM HEPES, and 36 mM glucose). Both hippocampi were dissected under a dissecting microscope and cut at 400 μm on a Mcllwain tissue chopper. Slices were separated and placed on filter inserts with a 30 mm diameter and a 0.4 μm filter pore size (Millipore, Billerica, Mass.) held in 6-well culture plates. For the first 3 days, slices were kept in Neurobasal media with B-27 supplement, 1 mM glutamine, and 1% penicillin/streptomycin (Invitrogen). Subsequently, the media was changed every 3 days using media without antibiotics. Organotypic cultures were maintained in a humidified incubator at 37° C. in 5% O₂ and 5% CO₂ for 14 to 21 days.

Cell death and viability was determined on live slices using the fluorescent probes from Molecular Probes' Live/Dead Kit. After a 40 minute incubation in calcein AM (1:400) and ethidium homodimer (1:1000) in phosphate buffered saline, slices were visualized at 200× magnification with an inverted Nikon Diaphot 200 microscope using the Bio-Rad MRC-1024 Laser Scanning Confocal system (Hercules, Calif.). Random images of the neuronal fields were captured at the emission spectrum of each probe, and Scion Image software (Frederick, Md.) was used to quantify the number of live and dead cells. At least three areas including a neuronal field were imaged and used to determine the percent death for each slice. The percent death was calculated as the number of EthD-1 positive cells divided by the total number of EthD-1 and calcein AM positive cells.

Treatments

β-amyloid₁₋₄₂ and reverse Aβ (Aβ₄₂₋₁) were obtained from BACHEM (Torrance, Calif.). These peptides were dissolved in 0.1% NH₃OH at 222 μM. For treatments, Aβ was aggregated at 50 μM in Neurobasal media with B-27 supplement and 1 mM glutamine at 37° C. for 24 hours. Then, the slice culture media was removed, and the slices were treated with 1 mL of 25 μM Aβ or vehicle for 24 hours. The protein fragments corresponding to the carboxyl-terminal region of sAPPα (synthesized by Sigma-Genosys, The Woodlands, Tex.) were added to a final concentration of 1 nM 48 hours prior to the addition of Aβ.

Results

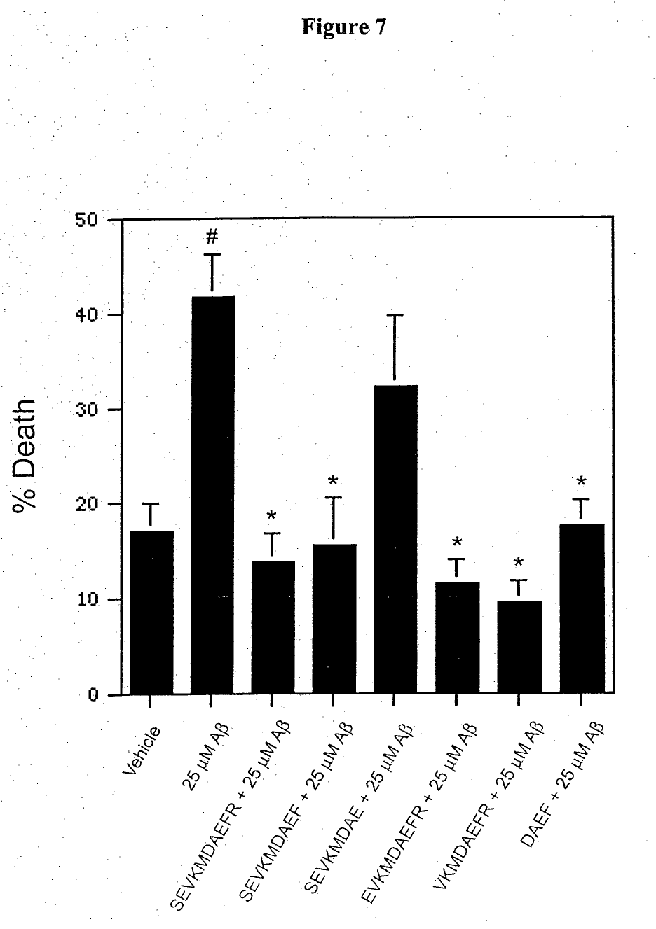

Organotypic hippocampal cultures were treated and incubated with ethidium homodimer (EthD-1) and calcein AM. The numbers of cells stained with each fluorescent probe were counted and expressed as the percent of cells that are EthD-1 positive and thus have lost membrane integrity (% Death, FIG. 7). Treatment with 25 μM Aβ resulted in a dramatic and significant increase in the percent death compared to treatment with vehicle. Pretreatment with 1 nM of a 10 amino acid fragment that corresponds to part of the carboxyl-terminal region of sAPPα (667-676 of APP770; SEVKMDAEFR (SEQ ID NO:1)) for 48 hours completely protected against Aβ-induced death. This protection was maintained when the carboxyl arginine was eliminated (SEVKMDAEF (SEQ ID NO:7)). However, elimination of both arginine and phenylalanine (SEVKMDAE (SEQ ID NO:22)) reversed the protection against Aβ. On the amino-terminal end, fragments with both the serine eliminated (EVKMDAEFR (SEQ ID NO:3)) and the serine and glutamic acid eliminated (VKMDAEFR (SEQ ID NO:4)) significantly protected against 25 μM Aβ treatment (FIG. 7). Pretreatment of hippocampal slices with 1 nM of a 4 amino acid fragment (DAEF) also was sufficient to protect against Aβ-induced cell death (FIG. 7).

### Example 10

### Polypeptide Protection In Vivo

The following experiments were performed to test the ability of sAPPα and a 10 amino acid fragment (SEVKMDAEFR (SEQ ID NO:1)) to activate cell survival pathways against Aβ-induced toxicity in vivo through stereotaxic injection into the hippocampi of mice. A scrambled form of the 10 amino acid fragment was used to demonstrate that pathway activation is dependent on the peptide sequence.

Stereotaxic Injections

Ten week old B6/SJL mice were stereotaxically injected on the right side of their hippocampi with sAPPα, a 10 amino acid fragment, or a scrambled 10-mer to an estimated 1 nM intra-hippocampal concentration. The contralateral sides were injected with artificial cerebrospinal fluid. Each injection consisted of 1.5 mL of solution delivered over the period of 10 minutes. 48 hours after surgery, the mice were perfused with cold PBS, and their hippocampi were dissected and stored in stabilizing solution for subsequent RNA extraction.

Quantitative PCR

RNA was extracted from hippocampal samples, DNase treated, and reverse transcribed. The cDNA was then used in quantitative real-time PCR performed with SYBR green on the Roche LightCycler system. Portions of each RNA sample were also used in no-RT traditional PCR reactions to validate the absence of DNA contamination.

Immunohistochemistry

48 hours after injection, mice were perfused with 4% PFA. The brains were harvested, hemisected, and fixed in 4% PFA overnight. They were then sunk in 30% sucrose for 24 hours, frozen, and sectioned. Sections were stained with cresyl violet for morphological verification of the injection site. Other sections were stained with goat anti-TTR, and biotinylated secondary antibody to show TTR protein expression, and ToPro3 as a nuclear marker. A control for nonspecific staining was done with goat IgG.

Results

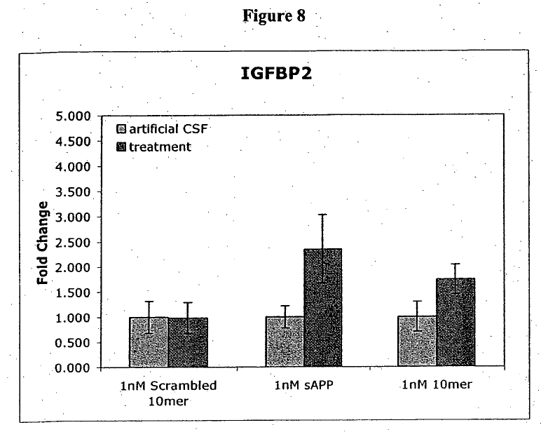

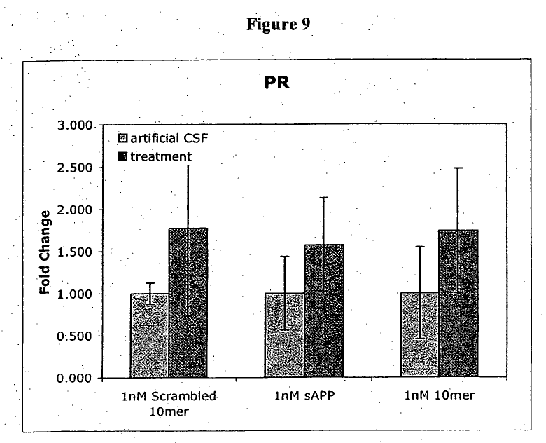

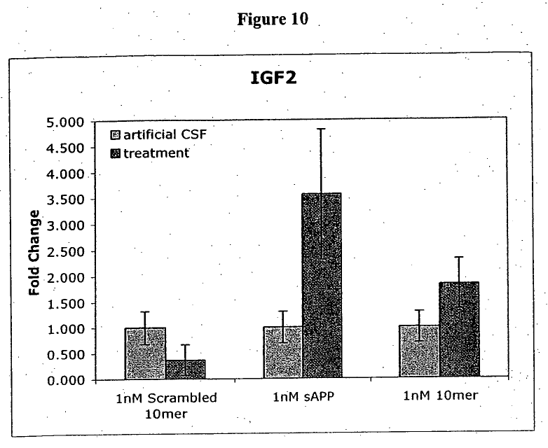

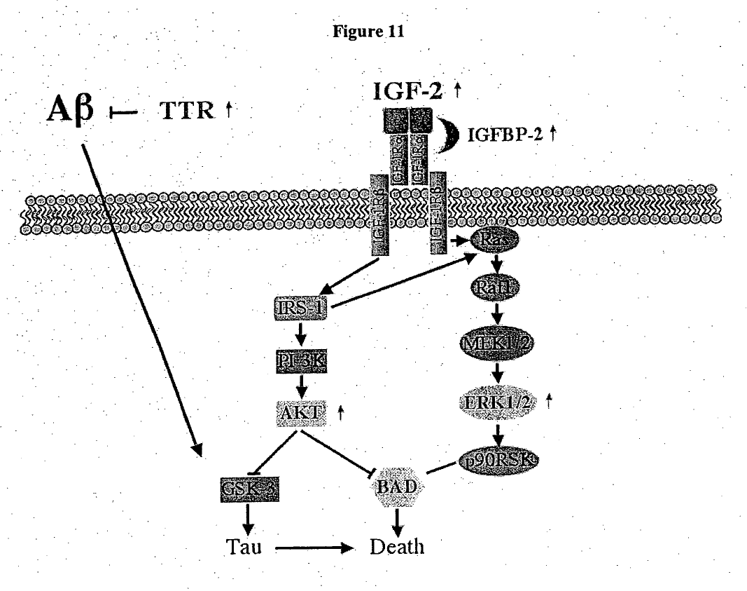

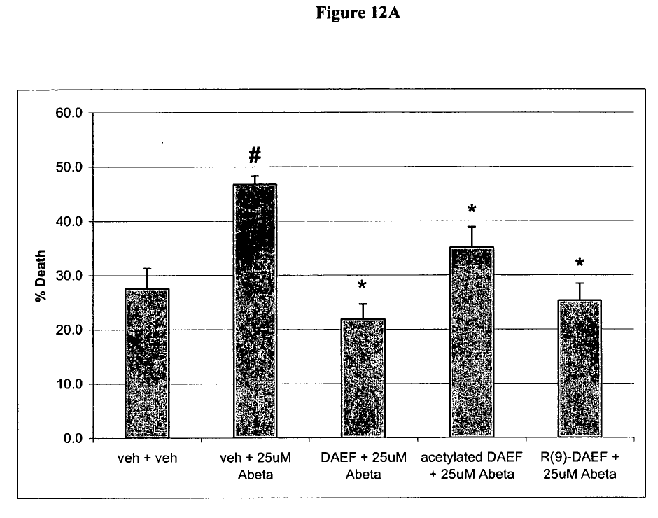

Both 1 nM sAPPα and the 10 amino acid fragment induced an increase in expression of the growth factors IGF2 and IGFBP2 (FIGS. 8, 9, and 10), which can protect against Aβ toxicity in vitro. In addition, sAPPα and the 10 amino acid fragment resulted in increased levels of TTR within treated hippocampi, which can protect against toxicity by sequestering Aβ. These results demonstrate that a neuroprotective pathway (see, e.g., FIG. 11) against Aβ toxicity can be activated in mice in vivo. These results also demonstrate that activation of a neuroprotective pathway can be used as a treatment in the prevention of Alzheimer's disease.

### Example 11

### (Prophetic)—Identifying Compounds In Vivo or In Vitro for the Ability to Reduce an Effect of Aβ

A test compound is applied to brain tissue that is in vivo or in vitro. The brain tissue also is contacted with Aβ that is either applied exogenously or expressed by cells within the brain tissue. Briefly, to obtain brain tissue for culture, brain tissue is removed in large, intact pieces (roughly 3 cm×2 cm×2 cm) by electrocautery as described in Example 8.

After contacting the brain tissue with the test compound, cell death and/or cell viability are determined with live slices using fluorescent probes from, for example, Molecular Probes' Live/Dead Kit. After a 40 minute incubation in calcein AM (1:400) and ethidium homodimer (1:1000) in phosphate buffered saline, slices are visualized, e.g. at 200× magnification with an inverted Nikon Diaphot 200 microscope using the Bio-Rad MRC-1024 Laser Scanning Confocal system (Hercules, Calif.). Random images are captured at the emission spectrum of each probe, and Scion Image software (Frederick, Md.) is used to quantify the number of live and dead cells. At least three areas are imaged and used to determine the percent death for each slice. The percent death is calculated as the number of EthD-1 positive cells divided by the total number of EthD-1 and calcein AM positive cells. In some cases, cell death is determined by TUNEL staining. Briefly, cortical slice cryosections are stained with terminal deoxynucleotidyl transferase-mediated, dUTP nick end labeling (TUNEL) and with an anti-NeuN antibody. Cells with DNA fragmentation are determined by the terminal deoxynucleotidyl transferase incorporation of fluorescein isothiocyanate (FITC)-12-dUTP into DNA (In Situ Cell Death Detection kit, Roche Biochem, Indianapolis, Ind.). At least 3 random images per slice are generated by confocal analysis at 600× magnification. The number of TUNEL positive, NeuN positive, and both TUNEL and NeuN positive cells are then quantified.

The percent cell death is compared to the percent cell death determined for control brain tissue contacted with Aβ in the absence of the test compound. If less cell death is observed with the brain tissue contacted with the test compound and Aβ as compared to that observed with brain tissue contacted with Aβ only, then the test compound can have the ability to reduce an effect of Aβ. A positive control for such a reduction is brain tissue contacted with a DAEF-containing polypeptide (e.g., SEVKMDAEFR (SEQ ID NO:1), SEVKMDAEF (SEQ ID NO:7), EVKMDAEFR (SEQ ID NO:3), VKMDAEFR (SEQ ID NO:4), or DAEF (SEQ ID NO:2)) and Aβ. Other controls include untreated brain tissue and brain tissue treated with β-amyloid₁₋₄₂ or reverse Aβ (Aβ₄₂₋₁).

The effectiveness of a test compound to reduce an effect of Aβ is determined by comparing the percent cell death observed with brain tissue contacted with the test compound and Aβ with the percent cell death observed with brain tissue contacted with a DAEF-containing polypeptide and Aβ. An equivalent or lesser level of cell death observed with brain tissue contacted with the test compound and Aβ as compared to that observed with brain tissue contacted with the DAEF-containing polypeptide and Aβ can indicate that the test compound is a potent inhibitor of an effect of Aβ.

### Example 12

### (Prophetic)—Identifying Compounds for the Ability to Reduce an Effect of Aβ

A test compound is applied to brain tissue that is in vivo or in vitro. Briefly, to obtain brain tissue for culture, brain tissue is removed in large, intact pieces (roughly 3 cm×2 cm×2 cm) by electrocautery as described in Example 8.

After contacting brain tissue with the test compound, the expression level of nucleic acid encoding a neuroprotective polypeptide such as TTR and IGF-2 is determined by measuring the polypeptide levels and/or mRNA levels. Briefly, an ELISA is used to measure the level of neuroprotective polypeptide expression, while RT-PCR or expression array technology (e.g., arrays available from Affymetrix (Santa Clara, Calif.)) is used to measure the level of mRNA expression for nucleic acid encoding a neuroprotective polypeptide.

The level of polypeptide or mRNA expression is compared to the polypeptide or mRNA expression level determined for control brain tissue not contacted with the test compound. If less polypeptide or mRNA expression is observed with the brain tissue contacted with the test compound as compared to that observed with control brain tissue, then the test compound can have the ability to reduce an effect of Aβ. A positive control for such a reduction is brain tissue contacted with a DAEF-containing polypeptide (e.g, SEVKMDAEFR (SEQ ID NO:1), SEVKMDAEF (SEQ ID NO:7), EVKMDAEFR (SEQ ID NO:3), VKMDAEFR (SEQ ID NO:4), or DAEF (SEQ ID NO:2)). Other controls include untreated brain tissue.

The effectiveness of a test compound to increase the expression level of nucleic acid encoding a neuroprotective polypeptide is determined by comparing the expression level observed with brain tissue contacted with the test compound with the expression level observed with brain tissue contacted with a DAEF-containing polypeptide. An equivalent or greater level of expression observed with brain tissue contacted with the test compound as compared to that observed with brain tissue contacted with the DAEF-containing polypeptide can indicate that the test compound is a potent activator of neuroprotective polypeptide expression.

### Example 13

### Evaluation of Polypeptides

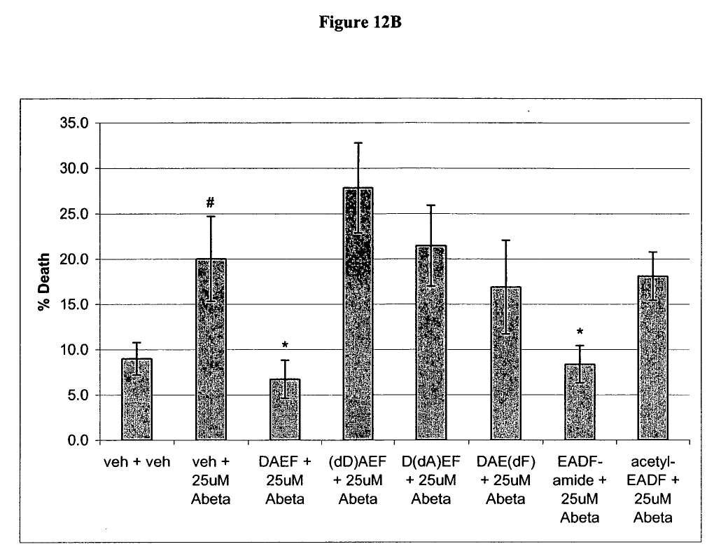

FIGS. 12A and B are a set of bar graphs showing percent cell death under a variety of treatment conditions. Referring to FIG. 12A, the tetrapeptide DAEF alone and with modifications on its NH₂-terminal end shows protection against Aβ-induced death in hippocampal slice cultures.

The percent death for each treatment was quantified in neuronal fields of live hippocampal slices by counting the number of membrane-permeable, EthD-1 positive cells as well as the number of live cells that stained positively with calcein AM. Data are expressed as mean±SEM (n=3−5 slices per treatment). Aβ results in a significant increase in the percent death while 1 nM of the tetrapeptide (DAEF) protects against the Aβ-induced toxicity. Acetylated DAEF (SEQ ID NO:2) demonstrates significant protection against Aβ. In addition, 9 arginines added to the NH2-terminal end of the tetrapeptide (R(9)-DAEF) significantly protect against Aβ-induced cell death. Still referring to FIG. 12A, #p-value<0.01 compared to vehicle; *p-value<0.05 compared to 25 μM Aβ; unpaired, two-tailed t-test.

Results

Organotypic hippocampal cultures were treated and incubated with ethidium homodimer (EthD-1) and calcein AM. Ethidium homodimer (EthD-1) is a membrane impermeable DNA-binding dye that is excluded from live cells with an intact plasma membrane. Calcein AM is a cell-permeant dye that fluoresces in live cells with a functional intracellular esterase. Here the numbers of cells stained with each fluorescent probe were counted and expressed as the percent of cells that are EthD-1 positive and thus have lost membrane integrity (% Death, Figure). Treatment with 25 μM Aβ resulted in a dramatic and significant increase in the percent death compared to treatment with vehicle. Pretreatment with 1 nM of the tetramer for 48 h completely protected against Aβ-induced death. This protection was maintained when the NH₂-terminal end is acetylated (acetyl-DAEF) though this molecule did not protect to the same degree as the unmodified tetrapeptide. Finally, the tetrapeptide modified to possess 9 arginines on its NH₂-terminal end (R(9)-DAEF) also significantly protected against Aβ treatment.

Experimental Procedures

Hippocampal Organotypic Cultures

Pups from C57B6/SJL mice were anaesthetized and decapitated at postnatal day 15. The brain was removed and placed in ice-cold dissecting medium (50% Minimum Essential Medium (Invitrogen, Carlsbad, Calif.), 50% Hank's Balanced Salt Solution (Invitrogen), 25 mM HEPES, and 36 mM glucose). Both hippocampi were dissected under a dissecting microscope and cut at 400 μm on a McIlwain tissue chopper. Slices were separated and placed on filter inserts with a 30 mm diameter and a 0.4 μm filter pore size (Millipore, Billerica, Mass.) held in 6-well culture plates. For the first 3 days slices were kept in Neurobasal media with B-27 supplement, 1 mM glutamine, and 1% penicillin/streptomycin (Invitrogen). Subsequently, the media was changed every 3 days using media without antibiotics. Organotypic cultures were maintained in a humidified incubator at 37° C. in 5% O₂ and 5% CO₂ for 14 to 21 days.

Cell death and viability was determined on live slices using the fluorescent probes from Molecular Probes' Live/Dead Kit. After a 40 min incubation in calcein AM (1:400) and ethidium homodimer (1:1000) in phosphate buffered saline, slices were visualized at 200× magnification with an inverted Nikon Diaphot 200 microscope using the Bio-Rad MRC-1024 Laser Scanning Confocal system (Hercules, Calif.). Random images of the neuronal fields were captured at the emission spectrum of each probe, and Scion Image software (Frederick, Md.) was used to quantify the number of live and dead cells. At least three areas including a neuronal field were imaged and used to determine the percent death for each slice. The percent death was calculated as the number of EthD-1 positive cells divided by the total number of EthD-1 and calcein AM positive cells.

Treatments

β-amyloid₁₋₄₂ and reverse Aβ (Aβ₄₂₋₁) were obtained from BACHEM (Torrance, Calif.). These peptides were dissolved in 0.1% NH₃OH at 222 μM. For treatments, Aβ was aggregated at 50 μM in Neurobasal media with B-27 supplement and 1 mM glutamine at 37° C. for 24 h. Then the slice culture media was removed and the slices were treated with 1 mL of 25 μM Aβ or vehicle for 24 h. The peptides and modified peptides (synthesized by Sigma-Genosys, The Woodlands, Tex.) were added to a final concentration of 1 nM 48 h prior to the addition of Aβ.

FIG. 12B shows the percent death for each treatment was quantified in neuronal fields of live hippocampal slices by counting the number of membrane-permeable, EthD-1 positive cells as well as the number of live cells that stained positively with calcein AM. Data are expressed as mean±SEM (n=3-5 slices per treatment). Aβ results in a significant increase in the percent death while 1 nM of the tetrapeptide (DAEF, SEQ ID NO:2) protects against the Aβ-induced toxicity. Tetrapeptide compounds containing a D-isomer of the D [(dD)AEF], A [D(dA)EF], or F [DAE(dF)] do not significantly protect against Aβ-induced death. A compound with the amino acids aspartic acid and glutamic acid switched and an amide group attached to the COOH-terminus (EADF (SEQ ID NO:5)-amide) significantly protects against Aβ-induced death while the EADF (SEQ ID NO:5) sequence with an acetyl group on the NH₂-terminus (acetyl-EADF (SEQ ID NO:5)) does not. Referring to FIG. 12B, #p-value<0.05 compared to vehicle; *p-value<0.05 compared to 25 μM Aβ; unpaired, two-tailed t-test.

Results

Organotypic hippocampal cultures were treated and incubated with ethidium homodimer (EthD-1) and calcein AM. Ethidium homodimer (EthD-1) is a membrane impermeable DNA-binding dye that is excluded from live cells with an intact plasma membrane. Calcein AM is a cell-permeant dye that fluoresces in live cells with a functional intracellular esterase. Here the numbers of cells stained with each fluorescent probe were counted and expressed as the percent of cells that are EthD-1 positive and thus have lost membrane integrity (% Death, Figure). Treatment with 25 μM Aβ resulted in a dramatic and significant increase in the percent death compared to treatment with vehicle. Pretreatment with 1 nM of the tetramer for 48 h completely protected against Aβ-induced death. This protection was eliminated by substituting D-isomers for amino acids D, A, and F.

Experimental Procedures

Hippocampal Organotypic Cultures

Pups from C57B6/SJL mice were anaesthetized and decapitated at postnatal day 15. The brain was removed and placed in ice-cold dissecting medium (50% Minimum Essential Medium (Invitrogen, Carlsbad, Calif.), 50% Hank's Balanced Salt Solution (Invitrogen), 25 mM HEPES, and 36 mM glucose). Both hippocampi were dissected under a dissecting microscope and cut at 400 μm on a McIlwain tissue chopper. Slices were separated and placed on filter inserts with a 30 mm diameter and a 0.4 μm filter pore size (Millipore, Billerica, Mass.) held in 6-well culture plates. For the first 3 days slices were kept in Neurobasal media with B-27 supplement, 1 mM glutamine, and 1% penicillin/streptomycin (Invitrogen). Subsequently, the media was changed every 3 days using media without antibiotics. Organotypic cultures were maintained in a humidified incubator at 37° C. in 5% O₂ and 5% CO₂ for 14 to 21 days.

Cell death and viability was determined on live slices using the fluorescent probes from Molecular Probes' Live/Dead Kit. After a 40 min incubation in calcein AM (1:400) and ethidium homodimer (1:1000) in phosphate buffered saline, slices were visualized at 200× magnification with an inverted Nikon Diaphot 200 microscope using the Bio-Rad MRC-1024 Laser Scanning Confocal system (Hercules, Calif.). Random images of the neuronal fields were captured at the emission spectrum of each probe, and Scion Image software (Frederick, Md.) was used to quantify the number of live and dead cells. At least three areas including a neuronal field were imaged and used to determine the percent death for each slice. The percent death was calculated as the number of EthD-1 positive cells divided by the total number of EthD-1 and calcein AM positive cells.

Treatments

β-amyloid₁₋₄₂ and reverse Aβ (Aβ₄₂₋₁) were obtained from BACHEM (Torrance, Calif.). These peptides were dissolved in 0.1% NH₃OH at 222 μM. For treatments, Aβ was aggregated at 50 μM in Neurobasal media with B-27 supplement and 1 mM glutamine at 37° C. for 24 h. Then the slice culture media was removed and the slices were treated with 1 mL of 25 μM Aβ or vehicle for 24 h. The peptides and modified peptides (synthesized by UW Biotechnology Peptide Synthesis Facility, Madison, Wis.) were added to a final concentration of 1 nM 48 h prior to the addition of Aβ.

### Example 14

### Injection of Polypeptide into Hippocampus

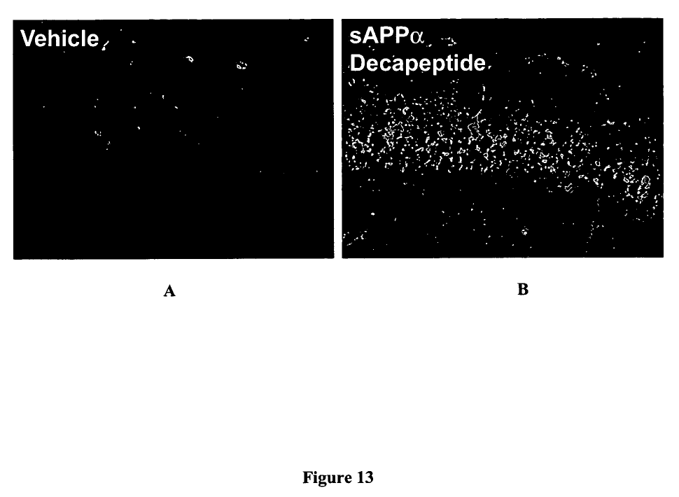

FIG. 13 is a set of stained micrographs showing stereotactic injection of sAPPα decamer into hippocampus induces expression of TTR in normal mouse brain. One nM of the decapeptide derived from sAPPα had previously been shown to protect organotypic hippocampal slice cultures against Aβ toxicity. Injection of 1.5 μl of 15.3 nM solution prepared in artificial cerebral spinal fluid (CSF) was performed to approximate a final concentration of 1 nM in the hippocampus. The contralateral side was injected with only artificial CSF and used as control. Immunohistochemistry for TTR demonstrated that injection of the decapeptide increased the amount of TTR in and around the CA1 hippocampal neurons (FIG. 13B) compared to the contralateral CSF injected hippocampus (FIG. 13A). The DNA binding dye ToPro3 was used to visualize nuclei (blue). Preliminary data also demonstrated increased TTR with injection of sAPPα and the tetramer.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

We claim:

1. An isolated peptide useful for reducing or preventing the effects of Aβ, wherein the peptide comprises the following formula,
A-B—C-D
wherein A is selected from the group consisting of amino acid residues D, E, N and Q;
wherein B is selected from the group consisting of amino acid residues A, T, S, G and P;
wherein C is selected from the group consisting of amino acid residues E, D, N and Q; and
wherein D is selected from the group consisting of amino acid residues F and Y;
wherein the peptide reduces or prevents the effects of Aβ in a mammalian cell and wherein the peptide is between 4-16 residues in length.

2. The peptide of claim 1 consisting essentially of A-B—C-D.

3. The peptide of claim 1 additionally comprising a polypeptide stabilizing unit.

4. The peptide of claim 1 additionally comprising amino acid residues at either the carboxy or amino terminal such that the formula of the peptide is
X-A-B—C-D-Z
wherein X and Z are residues from the sAPPα protein sequence contiguous with DAEF.

5. The peptide of claim 4, wherein the sAPPα residues are selected from 585-595 and 601-600 of sAPPα.

6. The peptide of claim 4, wherein A-B—C-D is flanked by the APP Swedish mutation wherein X comprises residues N-L adjacent to A.

7. The peptide of claim 3, wherein the peptide is selected from the group consisting of DAEF (SEQ ID NO:2), EVKMDAEFR (SEQ ID NO:3), VKMDAEFR (SEQ ID NO:4), EADF (SEQ ID NO:2), SEVKMDAEFR (SEQ ID NO:7), R₉DAEF (SEQ ID NO:2), MEADF (SEQ ID NO:8), EADFR (SEQ ID NO:9), EAEF (SEQ ID NO:10), and acDAEF (SEQ ID NO:2).

8. The peptide of claim 3, wherein the peptide is selected from the group consisting of PPDAEFPP (SEQ ID NO:12), PDAEFPP (SEQ ID NO:13), PPDAEFP (SEQ ID NO:14) and PDAEFP (SEQ ID NO:15).

9. An isolated peptide useful for reducing and preventing the effects of Aβ, wherein the peptide comprises an isolated 4-16 residue segment of the sAPPα sequence comprising DAEF (SEQ ID NO:2) (residues 597-600).

10. The peptide of claim 1,
Wherein A is selected from amino acid residues D and E;
Wherein B is amino acid residue A;
Wherein C is selected from amino acid residues E and D; and
Wherein D amino acid residue is F

11. The peptide of claim 1 additionally comprising a pharmaceutically acceptable carrier.

12. The peptide of claim 3 additionally comprising a pharmaceutically acceptable carrier.

13. The peptide of claim 9 additionally comprising a pharmaceutically acceptable carrier.

14. A method of reducing or preventing the effects of Aβ in a mammalian cell comprising the step of supplying a mammalian cell with an effective amount of the peptide of claim 1 such that the effects of Aβ are reduced or prevented.

15. A method of reducing or preventing the effects of Aβ in a mammalian cell comprising the step of supplying a mammalian cell with an effective amount of the peptide of claim 3 such that the effects of Aβ are reduced or prevented.

16. A method of reducing or preventing the effects of Aβ in a mammalian cell comprising the step of supplying a mammalian cell with an effective amount of the peptide of claim 8 such that the effects of Aβ are reduced or prevented.

17. The method of claim 14, wherein the effect of Aβ is Aβ-induced tau phosphorylation.

18. The method of claim 14, wherein the effect is neuronal cell death.

19. The method of claim 14, wherein the peptide is supplied to an AD patient.

20. A method of preventing neuronal cell death comprising the step of supplying a mammalian cell with an effective amount of peptide of claim 1 such that neuronal cell death is prevented.

21. A method of preventing neuronal cell death comprising the step of supplying a mammalian cell with an effective amount of peptide of claim 3 such that neuronal cell death is prevented.

22. A method of preventing neuronal cell death comprising the step of supplying a mammalian cell with an effective amount of peptide of claim 9 such that neuronal cell death is prevented.

23. The method of claim 20, wherein the peptide is supplied to an AD patient.

24. A method of identifying compounds having the ability to reduce the effect of Aβ comprising the steps of contacting brain tissue with a test compound in the presence of an Aβ peptide and measuring the effects of Aβ.

25. The method of claim 24 additionally comprising the step of comparing the test compound's ability to reduce the effect of Aβ to the ability of the peptide of claim 1 to reduce the effect of Aβ.

26. The method of claim 25 wherein the peptide is DAEF (SEQ ID NO:2) or SEVKMDAEFR (SEQ ID NO:1).


SEQUENCE LISTING (24 sequences)

SEQ ID NO: 1 - protein, 10 aa, Artificial
  Synthetic peptide.
SEVKMDAEFR

SEQ ID NO: 2 - protein, 4 aa, Artificial
  Synthetic peptide.
DAEF

SEQ ID NO: 3 - protein, 9 aa, Artificial
  Synthetic peptide.
EVKMDAEFR

SEQ ID NO: 4 - protein, 8 aa, Artificial
  Synthetic peptide.
VKMDAEFR

SEQ ID NO: 5 - protein, 4 aa, Artificial
  Synthetic peptide.
EADF

SEQ ID NO: 6 - protein, 13 aa, Artificial
  Synthetic peptide.
RRRRRRRRRDAEF

SEQ ID NO: 7 - protein, 9 aa, Artificial
  Synthetic peptide.
SEVKMDAEF

SEQ ID NO: 8 - protein, 5 aa, Artificial
  Synthetic peptide.
MEADF

SEQ ID NO: 9 - protein, 5 aa, Artificial
  Synthetic peptide.
EADFR

SEQ ID NO: 10 - protein, 4 aa, Artificial
  Synthetic peptide.
EAEF

SEQ ID NO: 11 - protein, 9 aa, Artificial
  Synthetic peptide.
RKKRRQRRR

SEQ ID NO: 12 - protein, 8 aa, Artificial
  Synthetic peptide.
PPDAEFPP

SEQ ID NO: 13 - protein, 7 aa, Artificial
  Synthetic peptide.
PDAEFPP

SEQ ID NO: 14 - protein, 7 aa, Artificial
  Synthetic peptide.
PPDAEFP

SEQ ID NO: 15 - protein, 6 aa, Artificial
  Synthetic peptide.
PDAEFP

SEQ ID NO: 16 - DNA, 21 bp, Artificial
  Synthetic oligonucleotide.
aatccaaatgtcctctgatgg

SEQ ID NO: 17 - DNA, 21 bp, Artificial
  Synthetic oligonucleotide.
aactggacaccaaatcgtact

SEQ ID NO: 18 - DNA, 21 bp, Artificial
  Synthetic oligonucleotide.
aaggggatagagatgtgagag

SEQ ID NO: 19 - DNA, 21 bp, Artificial
  Synthetic oligonucleotide.
aaattatgtggtaattctgca

SEQ ID NO: 20 - DNA, 21 bp, Artificial
  Synthetic oligonucleotide.
aacgactatcagcagctgaag

SEQ ID NO: 21 - DNA, 21 bp, Artificial
  Synthetic oligonucleotide.
aacagctggaacatggtggat

SEQ ID NO: 22 - protein, 8 aa, Artificial
  Synthetic peptide.
SEVKMDAE

SEQ ID NO: 23 - protein, 4 aa, Artificial
  Synthetic peptide.
EVKM

SEQ ID NO: 24 - protein, 4 aa, Artificial
  Synthetic peptide.
XXXX